(19) United States
(12) Patent Application Publication
(10) Pub. No.: US 2024/0165130 A1
(43) Pub. Date: May 23, 2024

(54) COMPOSITE DRUG PARTICLES AND USES THEREOF

(71) Applicant: THE REGENTS OF THE UNIVERSITY OF MICHIGAN, Ann Arbor, MI (US)

(72) Inventors: Omolola Eniola Adefeso, Ann Arbor, MI (US); Michael L. Felder, Ann Arbor, MI (US); Hanieh Safari, Ann Arbor, MI (US); Daniel Kupor, Ann Arbor, MI (US)

(21) Appl. No.: 18/551,809

(22) PCT Filed: Mar. 31, 2022
(86) PCT No.: PCT/US22/22848

§ 371 Date: Sep. 21, 2023

Related U.S. Application Data
(60) Provisional application No. 63/168,829, filed on Mar. 31, 2021.

(51) Int. Cl. A61K 31/575; A61K 9/16; A61K 31/573; A61P 35/00; A61P 43/00

(52) U.S. Cl. CPC A61K 31/575; A61K 9/1611; A61K 9/1617; A61K 9/1682; A61K 31/573; A61P 35/00; A61P 43/00

(57) ABSTRACT

Provided herein are particles comprising compounds having a steroid core structure, or salts or esters thereof. Also provided herein are compositions comprising the particles, and methods of using the particles, for example in methods of treating liver disorders or for fat reduction.

### CROSS-REFERENCE TO RELATED APPLICATIONS

This application claims priority to and the benefit of U.S. Provisional Patent Application No. 63/168,829, filed on Mar. 31, 2021, which is incorporated herein by reference in its entirety.

### TECHNICAL FIELD

Provided herein are composite particles comprising compounds having a steroid core structure, or salts or esters thereof. Also provided herein are compositions comprising the particles, and methods of using the particles, for example in methods of treating liver disorders or for fat reduction.

### BACKGROUND

Compounds having a steroid backbone, such as bile acids and corticosteroids, are useful in treating a wide variety of disorders. However, oral and subcutaneous administration of solubilized versions of these drugs may have limited efficacy and may impose unwanted side effects.

### SUMMARY

The present disclosure provides a composite particle comprising a compound having a steroid core structure, or a salt or ester thereof, wherein the composite particle has a length in at least one dimension of at least 100 nm.

In some embodiments, the composite particle further comprises transition metal ions. In some embodiments, the transition metal ions are selected from gold, silver, copper, platinum, palladium, nickel, and iron ions. In some embodiments, the transition metal ions are selected from gold, silver, copper, and iron ions. In some embodiments, the transition metal ions are gold ions (e.g., Au(III) ions) or iron ions (e.g., Fe(II) ions).

In some embodiments, the composite particle further comprises an acid. In some embodiments, the acid is selected from hydrochloric acid and salicylic acid.

In some embodiments, the compound having a steroid core structure is selected from the group consisting of testosterone, exemestane, formestane, mesterolone, fluoxymesterone, methyltestosterone, oxandrolone, oxymetholone, mestranol, norethindrone, danazol, gestrinone, levonorgestrel, lynestrenol, norgestrel, desogestrel, etonogestrel, tibolone, ethynodiol, cyproterone, megestrol, abiraterone, dienogest, mifepristone, drospirenone, spironolactone, estradiol, polyestradiol, estramustine, estrone, estropipate, progesterone, dydrogesterone, hydroxyprogesterone, medroxyprogesterone, segesterone, norelgestromin, norgestimate, cortisol, cortisone, fluorometholone, difluprednate, fludrocortisone, fluocinolone, loteprednol, methylprednisolone, prednicarbate, prednisolone, prednisone, triamcinolone, alclometasone, betamethasone, clobetasol, clobetasone, clocortolone, desoximetasone, dexamethasone, diflorasone, difluocortolone, fluticasone, halometasone, mometasone, rimexolone, amcinonide, budesonide, ciclesonide, deflazacort, desonide, flunisolide, fluocinonide, halcinonide, cholesterol, estradiol, hydrocortisone, diflucortolone, boldenone, nandrolone, altrenogest, stanozolol, osaterone, estriol, aglepristone, trilostane, flumethasone, deoxycorticosterone, alfaxalone, desoxycorticosterone, and isoflupredone, or a salt or an ester thereof, or any combination thereof.

In some embodiments, the compound having a steroid core structure is a bile acid. In some embodiments, the bile acid is selected from the bile acid is selected from cholic acid, deoxycholic acid, chenodeoxycholic acid, lithocholic acid, glycocholic acid, taurocholic acid, g ycodeoxycholic acid, taurodeoxycholic acid, glycochenodeoxycholic acid, taurochenodeoxycholic acid, glycolithocholic acid, taurolithocholic acid, ursodeoxycholic acid, g ycoursodeoxycholic acid, tauroursodeoxycholic acid, and obeticholic acid In some embodiments, the bile acid is selected from cholic acid and deoxycholic acid. In some embodiments, the compound having a steroid core structure is a salt of a bile acid. In some embodiments, the salt of the bile acid is selected from sodium cholate, sodium deoxycholate, sodium ursodeoxycholate, and sodium chenodeoxycholate.

In some embodiments, the compound haying a steroid core structure is a corticosteroid compound or a salt or ester thereof. In sonic embodiments, the corticosteroid compound is selected from hydrocortisone, dexamethasone, beclomethasone, ciclesonide, clobetasol, clobetasone, desonide, desoxytnethasone, desoxycorticosterone, dichlorisone, diflorasone, diflucortolone, fluclarolone, fludrocortisone, flumethasone, fluocinolone, fluocinonide, flucortine, fluocortolone, fluprednidene, flurandrenolone, halcinonide, halometasone, methylprednisolone, triamcinolone, cortisone, cortodoxone, flucetonide, fluradrenalone, medrysone, alclometasone, amciafel, amcinafide, amcinonide, betamethasone, budesonide, chlorprednisone, clocortelone, clescinolone, difluprednate, ilucloronide, fluoromethalone, fluperolone, fluprednisolone, hydrocortamate, meprednisone, mometasone, paramethasone, prednisolone, prednisone, prednicarbate, and tixocortol, or a salt or an ester thereof. In some embodiments, the corticosteroid compound is selected from dexamethasone, methylprednisolone, and hydrocortisone, or a salt or an ester thereof.

In some embodiments, the composite particle has a length in at least one dimension of at least 1 μm.

In some embodiments, the composite particle has a hexagonal prism shape. In some embodiments, the hexagonal prism has a diagonal length of 2.5 μm to 10 μm. In some embodiments, the hexagonal prism has a height of 2.5 μm to 6.5 μm.

In some embodiments, the composite particle has a rod shape. In some embodiments, the rod has a length of 2.5 μm to 100 μm. In some embodiments, the rod has a length of 10 μm to 50 μm.

In some embodiments, the particle consists essentially of: (i) the compound having a steroid core structure or a salt or ester thereof, and (ii) transition metal ions and/or an acid. In some embodiments, the particle consists essentially of a bile acid or salt or ester thereof and gold ions (e.g., Au(III) ions).

In some embodiments, the particle is essentially free of transition metal nan.oparticles (e.g., gold nanoparticles).

The present disclosure also provides a composition comprising a plurality of composite particles described herein. In some embodiments, the composition further comprises a pharmaceutically acceptable carrier.

The present disclosure also provides a method of making a plurality of composite particles, comprising:
(a) providing a first solution comprising a transition metal salt in ethyl acetate;
(b) mixing the first solution with water to form a first emulsion;
(c) mixing the first emulsion with a second solution, wherein the second solution comprises a compound having a steroid core structure or a salt or ester thereof, to form a final mixture and thereby form the composite particles.

The present disclosure also provides a method of making a plurality of composite particles, comprising:
(a) providing a first solution comprising a compound having a steroid core structure or a salt or ester thereof in ethyl acetate;
(b) mixing the first solution with a transition metal salt to form a second solution;
(c) mixing the second solution with water to form a first emulsion;
(d) mixing the first emulsion with a solution of a compound having a steroid core structure or a salt or ester thereof in water to form a final mixture and thereby form the composite particles.

The present disclosure also provides a method of making a plurality of composite particles, comprising:
(a) providing a first solution comprising a transition metal salt in water;
(b) mixing the first solution with ethyl acetate to form a first emulsion;
(c) mixing the first emulsion with a solution of a compound having a steroid core structure or a salt or ester thereof in water to form a final mixture and thereby form the composite particles.

The present disclosure also provides a method of making a plurality of composite particles, comprising:
(a) providing a first solution comprising an acid, a compound having a steroid core structure or a salt or ester thereof, and water;
(b) mixing the first solution with ethyl acetate to form a first emulsion;
(c) mixing the first emulsion with a solution of an acid in water to form a final mixture and thereby form the composite particles.

The present disclosure also provides a method of making a plurality of composite particles, comprising:
(a) providing a first solution comprising an acid in ethyl acetate;
(b) mixing the first solution with a second solution comprising a compound having a steroid core structure or a salt or ester thereof and water, to form a first emulsion;
(c) mixing the first emulsion with a third solution comprising a compound having a steroid core structure or a salt or ester thereof and water, to form a final mixture and thereby form the composite particles.

In some embodiments of the methods disclosed herein, the compound having a steroid core structure is a bite acid or a salt or an ester thereof. In some embodiments, the compound having a steroid core structure is selected from sodium cholate, sodium deoxycholate, sodium ursodeoxycholate, and sodium chenodeoxycholate. In some embodiments, the compound having a steroid core structure is a corticosteroid compound or a salt or an ester thereof. In some embodiments, the corticosteroid compound is selected from dexamethasone, methylprednisolone, and hydrocortisone, or a salt or an ester thereof. In some embodiments, the transition metal salt is selected from a gold(III) salt, a silver(I) salt, a copper(II) salt, a platinum(II) salt, a palladium(II) salt, a nickel(II) salt, an iron(II) salt, and an iron(III) salt. In some embodiments, the transition metal salt is selected from a gold(III) salt, a silver(I) salt, a copper(II) salt, and an iron(II) salt. In some embodiments, the transition metal salt is gold(III) chloride or iron(II) sulfate.

In some embodiments of the methods disclosed herein, the method further comprises a step of stirring the final mixture for about 15 seconds to about 15 minutes. In some embodiments, the method further comprises removing the solvents from the final mixture. In some embodiments, the method further comprises separating the composite particles from the final mixture. In some embodiments, the method is conducted entirely at ambient temperature. In some embodiments, the final mixture does not comprise a reducing agent.

The present disclosure also provides a method of treating a liver disease or a peroxisomal disorder in a subject m need of treatment, comprising administering to the subject a therapeutically effective amount of a composition described herein (e.g., a composition comprising a plurality of composite particles described herein). In some embodiments, the liver disease is a bile acid synthesis disorder or primary biliary cholangitis. In some embodiments, the liver disease is a bile acid synthesis disorder due to a single enzyme defect. In some embodiments, the peroxisomal disorder is a Zellweger spectrum disorder.

The present disclosure also provides a method of non-surgical removal of a localized fat deposit in a subject, comprising contacting the deposit with an effective amount of a composition described herein (e.g., a composition comprising a plurality of composite particles described herein).

The present disclosure also provides a method of reducing a subcutaneous fat deposit in a subject in need thereof, comprising administering locally to the subcutaneous fat deposit in the subject an effective amount of a composition described herein (e.g., a composition comprising a plurality of composite particles described herein).

The present disclosure also provides a method of treating cancer in a subject in need thereof, comprising administering to the subject an effective amount of a composition described herein (e.g., a composition comprising a plurality of composite particles described herein). In some embodiments, the cancer is colorectal cancer or gastric cancer.

The present disclosure also provides a method of reducing the proliferation of cancer cells, comprising contacting the cells with an effective amount of a composition described herein (e.g., a composition comprising a plurality of composite particles described herein). In some embodiments, the cancer cells are colorectal cancer cells or gastric cancer cells.

The present disclosure also provides a method of treating a disorder selected from the group consisting of endocrine disorders, rheumatic disorders, collagen diseases, dermatologic diseases, allergic states, ophthalmic diseases, respiratory diseases, hematologic disorders, neoplastic diseases, gastrointestinal diseases, nervous system disorders, inflammatory disorders, and renal diseases, comprising administering to the subject a therapeutically effective amount of a composition described herein (e.g., a composition comprising a plurality of composite particles described herein).

The present disclosure also provides uses of the particles and compositions described herein (e.g., use for removal of a localized fat deposit, use for reducing a subcutaneous fat deposit in a subject, use in the treatment of cancer such as colorectal cancer, use in reducing the proliferation of cancer cells, use in the treatment of disorders selected from endocrine disorders, rheumatic disorders, collagen diseases, dermatologic diseases, allergic states, ophthalmic diseases, respiratory diseases, hematologic disorders, neoplastic diseases, gastrointestinal diseases, nervous system disorders, inflammatory disorders, and renal diseases, etc.).

### BRIEF DESCRIPTION OF THE DRAWINGS

FIGS. 1A-1D show scanning electron microscopy (SEM) images of composite particles prepared with different compounds having a steroid core structure: (FIG. 1A) cholate; (FIG. 1B) deoxycholate; (FIG. 1C) methylprednisolone; and (FIG. 1D) hydrocortisone; all prepared with gold(III) chloride.

FIG. 2 shows energy-dispersive spectroscopy (EDS) data for cholate/gold ion particles prepared according to methods disclosed herein.

FIGS. 3A-3B show: (FIG. 3A) a brightfield microscopy image of rhodamine-loaded cholate particles containing gold ions; and (FIG. 3B) a fluorescence microscopy image of the same rhodamine-loaded cholate particles.

FIGS. 4A-4B show an SEM image (FIG. 4A) and EDS data (FIG. 4B) for composite particles prepared with dexamethasone phosphate and iron (II) sulfate heptahydrate.

FIG. 5 shows degradation data for composite particles prepared with dexamethasone phosphate and iron (II) sulfate heptahydrate

FIGS. 6A-6B show SEM images of composite particles prepared from hydrochloric acid and: (FIG. 6A) sodium cholate; (FIG. 6B) sodium deoxycholate; sodium chenodeoxycholate (FIG. 6C); and sodium ursodeoxycholate (FIG. 6D).

FIGS. 7A-7B show SEM images of composite particles prepared from hydrochloric acid and: methylprednisolone succinate (FIG. 7A); and hydrocortisone succinate (FIG. 7B).

FIGS. 8A-8F show SEM images of composite particles prepared from salicylic acid and: (FIG. 8A): sodium cholate; (FIG. 8B) sodium deoxycholate; (FIG. 8C) methylprednisolone succinate; (FIG. 8D) sodium chenodeoxycholate; (FIG. 8E) hydrocortisone succinate; and (FIG. 8F) sodium ursodeoxycholate.

FIG. 9 shows UV-visible spectrometry data demonstrating salicylic acid release from deoxycholate particles.

FIGS. 10A-10B show SEM images of cholate/gold ion particles when exposed to a degradation protocol as detailed in Example 2: (FIG. 10A) cholate/gold ion particles prepared according to methods disclosed herein on days 0, 1, 3, 5, 10, and 15 of a degradation protocol; and (FIG. 10B) comparison cholate/gold(0) nanoparticle particles prepared according to a previously-disclosed method, on days 0, 1. 3, 5, 10, and 15 of a degradation protocol.

FIG. 11 shows data for the release of cholate from particles subjected to a degradation process.

FIG. 12 shows dynamic light scattering (DLS) data showing the presence of gold nanoparticles following degradation of cholate particles prepared according to a previously disclosed method.

FIG. 13 shows UV-visible spectrometry data for the detection of gold nanoparticles following degradation of deoxycholate particles prepared according to a method disclosed herein, and deoxycholate particles prepared according to a previously disclosed method.

FIG. 14 shows images of samples subjected to a gold nanoparticle formation process.

FIGS. 15A-15B show images of samples in which beef fat was incubated with particles disclosed herein, resulting in cell lysis: (FIG. 15A) negative and positive controls; (FIG. 15B) negative control and samples incubated with the indicated particles; in each case, arrows point to lipid droplets.

FIG. 16 shows results of cell viability assays when HT-29 colon cancer cells were incubated with composite particles described herein.

### DETAILED DESCRIPTION

The present disclosure relates to particles that may be used for controlled release of compounds having a steroid core structure, or their salts. The particles may be used for a variety of medical and dermatological treatments, such as treatments of liver disorders and for non-surgical removal of localized fat deposits. The particles can also be used for the treatment of cancer or in a method of reducing proliferation of cancer cells.

Previous work in fabrication of composite particles required transition metal (e.g., gold) nanoparticle formation to produce particles composed of bile salts. See, e.g., International Patent Publication No. WO 2021/011753. The nanoparticles were present in the sample as a result of the fabrication process, in which transition metal precursors were reduced to form elemental transition metal nanoparticles in situ. A heating step was used to effect reduction of the transition metal ions, and particles formed in solution alongside the transition metal nanoparticles.

Composite particles disclosed herein do not include transition metal nanoparticles. Rather, the particles are fabricated from a solution containing either transition metal ions or an acid, and a reduction step is not required to template particle formation. Without wishing to be limited by theory, the particles disclosed herein are believed to form as a result of the establishment of an appropriate pH in the solution or emulsion, which can be achieved either using a solution of an acid or of a transition metal salt that forms an acidic solution when dissolved in water. In some embodiments, (e.g., when transition metal ions are used), particles may form as a result of formation of a complex between transition metal ions and a compound having a steroid backbone. The particles can be prepared in several ways, none of which involve a reducing agent or a heating step, such that transition metal nanoparticles do not form.

### Composite Particles

In one aspect, the disclosure provides a composite particle comprising a compound having a steroid core structure, or a salt or ester thereof, wherein the composite particle has a length in at least one dimension of at least 100 nm. In some embodiments, the particle further comprises transitional metal ions (e.g., from a transition metal salt). In some embodiments, the particle further comprises an acid.

The composite particles include a compound having a steroid core structure, as shown below, with the conventional numbering on the perhydrocyclopenta[a]phenanthrene core:

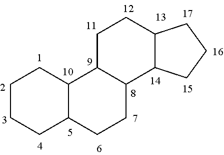 [SMILES: C1CCC2C(C1)CCC1C3CCCC3CCC21]

The steroid core structure can be fully saturated as shown above, or can include one or more double bonds. The core structure can include one or more alkyl functional groups; for example, steroid compounds contain methyl groups at the C10 and C13 positions, and often contain an alkyl group (or a functionalized alkyl group) at C17. The core structure can also include one or more hydroxy or oxo groups; for example, steroids and sterols have an oxo or hydroxy group at C3.

In some embodiments, the compound having a steroid core structure is selected from the group consisting of testosterone (e.g., testosterone enanthate, testosterone cypionate, or testosterone undecanoate), exemestane, formestane, mesterolone, fluoxymesterone, methyltestosterone, oxandrolone, oxymetholone, mestranol, norethindrone, danazol, gestrinone, levonorgestrel, lynestrenol, norgestrel, desogestrel, etonogestrel, tibolone, ethynodiol ethynodiol diacetate), cyproterone, megestrol (e.g., megestrol acetate), abiraterone (e.g., abiraterone acetate), dienogest, mifepristone, drospirenone, spironolactone, estradiol, polyestradiol phosphate, estramustine estramustine phosphate), estrone, estropipate, progesterone, dydrogesterone, hydroxyprogesterone hydroxyprogesterone caproate), medroxyprogesterone medroxyprogesterone acetate), segesterone (e.g., segesterone acetate), norelgestromin, norgestimate, cortisol, cortisone, fluorometholone, difluprednate, fludrocortisone (e.g., fludrocortisone acetate), fluocinolone (e.g., fluocinolone acetonide), loteprednol (e.g., loteprednol etabonate), methylprednisolone (e.g., tnethylprednisolone acetate or methylprednisolone succinate), prednicarbate, prednisolone (e.g., prednisolone sodium phosphate or prednisolone acetate), prednisone, triamcinolone (e.g., triamcinolone acetonide or triamcinolone hexacetonide), alclometasone (e.g., alclometasone diproprionate), betamethasone (e.g., betamethasone sodium phosphate, betamethasone benzoate, betamethasone dipropionate, betamethasone valerate, or betamethasone acetate), clobetasol (e.g., clobetasol propionate), clobetasone (e.g., clobetasone butyrate), clocortolone (e.g., clocortolone pivalate), desoximetasone, dexamethasone (e.g., dexamethasone phosphate or dexamethasone sodium phosphate), diflorasone (e.g., diflorasone diacetate), difluocortolone, fluticasone (fluticasone propionate or fluticasone furoate), halometasone, mometasone (e.g., mometasone furoate), rirnexolone, amcinonide, budesonide, ciclesonide, deflazacort, desonide, flunisolide, fluocinonide, halcinonide, cholesterol, estradiol valerate, hydrocortisone (e.g., hydrocortisone acetate, hydrocortisone buteprate, hydrocortisone butyrate, hydrocortisone succinate, or hydrocortisone valerate), diflucortolone diflucortolone valerate), boldenone (e.g., boldenone undecylenate), nandrolone, altrenogest, stanozolol, osaterone (e.g., osaterone acetate), estriol, aglepristone, trilostane, flumethasone, deoxycorticosterone, alfaxalone, desoxycorticosterone (e.g., desoxycorticosterone piyalate), and isoflupredone isoflupredone acetate), or any combination thereof.

In some embodiments, the composite particles comprise the compound having a steroid core structure or salt or ester thereof in an amount of about 70 wt % to about 99 wt %, or about 80 wt % to about 95 wt %. For example, in some embodiments the composite particles comprise the compound having a steroid core structure or salt or ester thereof in an amount of about 70 wt %, 71 wt %, 72 wt %, 73 wt %, 74 wt %, 75 wt %, 76 wt %, 77 wt %, 78 wt %, 79 wt %, 80 wt %, 81 wt %, 82 wt %, 83 wt %, 84 wt %, 85 wt %, 86 wt %, 87 wt %, 88 wt %, 89 wt %, 90 wt %, 91 wt %, 92 wt %, 93 wt %, 94 wt %, 95 wt %, 96 wt %, 97 wt %, 98 wt %, or 99 wt %, or any range therebetween.

In some embodiments, the compound having a steroid core structure is a bile acid, or a salt or ester thereof. In such embodiments, the disclosure provides a composite particle comprising a bile acid, or a salt or ester thereof, wherein the composite particle has a length in at least one dimension of at least 100 nm. In some embodiments, the composite further comprises transition metal ions and/or an acid. In some etnbodiments, the disclosure provides a composite particle comprising a bile acid, or a salt or ester thereof, and gold ions (e.g., Au(III) ions) or iron ions (e.g., Fe(II) ions). In some embodiments, the disclosure provides a composite particle comprising a bile acid, or a salt or ester thereof, and an acid (e.g., hydrochloric acid or salicylic acid).

Bile acids are steroid acids primarily found in bile, including both primary bile acids, which are synthesized by the liver, and secondary bile acids, which are synthesized from primary bile acids by bacteria in the colon. Bile acids and their salts help to solubilize lipids in the small intestine and regulate several hepatic, biliary, and intestinal functions; they have been proposed as therapeutic agents for treatment of different conditions including bile acid synthesis disorders and peroxisomal disorders, primary biliary cholangitis, primary sclerosing cholangitis, cardiornetabolic diseases, gallstones and bile duct stones, non-alcoholic fatty liver disease, type-2 diabetes, human immunodeficiency virus type 1 (HIV-1), acute pancreatitis, and cancer.

Orally administered formulations of certain bile acids have been approved by the United States Food and Drug Administration (FDA) for treatment of different bile synthesis disorders and liver dysfunctions. For example, cholic acid capsules are approved for treatment of bile acid synthesis disorders and peroxisomal disorders, and both ursodeoxycholic acid tablets and obeticholic acid tablets have been approved for treatment of primary biliary cholangitis. However, oral administration of these drugs may limit their bioavailability and can impose cytotoxicity risks through their membrane disruptive properties. Additionally, injectable deoxycholic acid is approved by the U.S. FDA for destruction of fat cells to reduce moderate-to-severe fat below the chin. However, numerous injections are required for effective treatment. Formulation of bile acids in the composite particles disclosed herein provide controlled or targeted delivery of bile acids to increase specificity while lowering side effects.

Bile acids may be conjugated with taurine or glycine. Exemplary bile acids include cholic acid, deoxycholic acid, chenodeoxycholic acid, lithocholic acid, glycocholic acid, taurocholic acid, glycodeoxycholic acid, taurodeoxycholic acid, glycochenodeoxycholic acid, taurochenodeoxycholic acid, glycolithocholic acid, taurolithocholic acid, ursodeoxycholic acid, glycoursodeoxycholic acid, and tauroursodeoxycholic acid. Bile acids also include semi-synthetic bile acids, such as obeticholic acid. In some embodiments, the bile acid is selected from cholic acid, deoxycholic acid, ursodeoxycholic acid, and chenodeoxycholic acid. In some embodiments, the bile acid is selected from cholic acid and deoxycholic acid. In some embodiments, the bile acid is cholic acid. In some embodiments, the bile acid is deoxycholic acid. In some embodiments, the composite particles comprise a combination of two or more bile acids or salts thereof.

The bile acid may be in the form of a salt. In some embodiments, the bile acid salt is a sodium or potassium salt. In some embodiments, the bile acid salt is a sodium salt. Accordingly, in some embodiments, the particle comprises a bile acid salt selected from sodium cholate, sodium deoxycholate, sodium ursodeoxycholate, and sodium chenodeoxycholate. In some embodiments, the particle comprises a bile acid salt selected from sodium cholate and sodium deoxycholate. In some embodiments, the bile acid salt is sodium cholate. In some embodiments, the bile acid salt is sodium deoxycholate. In some embodiments, the bile acid salt is sodium ursodeoxycholate. In some embodiments, the bile acid salt is sodium chenodeoxycholate.

In some embodiments, the composite particles comprise the bile acid or salt or ester thereof in an amount of about 70 wt % to about 99 wt %, or about 80 wt % to about 95 wt %. For example, in some embodiments the composite particles comprise the bile acid or salt or ester thereof in an amount of about 70 wt %, 71 wt %, 72 wt %, 73 wt %, 74 wt %, 75 wt %, 76 wt %, 77 wt %, 78 wt %, 79 wt %, 80 wt %, 81 wt %, 82 wt %, 83 wt %, 84 wt %, 85 wt %, 86 wt %, 87 wt o, 88 wt %, 89 wt %, 90 wt %, 91 wt %, 92 wt %, 93 wt %, 94 wt %, 95 wt %, 96 wt %, 97 wt %, 98 wt %, or 99 wt %, or any range therebetween.

In some embodiments, the compound having a steroid core structure is a corticosteroid, or a salt or ester thereof. In such embodiments, the disclosure provides a composite particle comprising a corticosteroid, or a salt or ester thereof, wherein the composite particle has a length in at least one dimension of at least 100 nm. In some embodiments, the composite particle further comprises transition metal ions and/or an acid. In some embodiments, the disclosure provides a composite particle comprising a corticosteroid, or a salt or ester thereof, and gold ions (e.g., Au(III) ions) or iron ions (e.g., Fe(II) ions).

Corticosteroids are a class of steroid hormones produced in the adrenal cortex of vertebrates, as well as synthetic analogs of such hormones. The two main categories of corticosteroids are glucocorticoids and mineralocorticoids. These compounds are involved in a wide range of physiological processes, including stress response, immune response, and regulation of inflammation, carbohydrate metabolism, protein catabolism, blood electrolyte levels, and behavior.

Corticosteroids are generally grouped into four classes: Group A (hydrocortisone type), including hydrocortisone (e.g., hydrocortisone acetate, hydrocortisone aceponate, hydrocortisone buteprate, hydrocortisone butyrate, hydrocortisone cypionate, or hydrocortisone succinate), cortisone (e.g., cortisone acetate), tixocortol (e.g., tixocortol pivalate), prednisolone, methylprednisolone (e.g., methylprednisolone succinate), and prednisone; Group B (acetonides and related compounds), including amcinonide, budesonide, desonide, fluocinolone (e.g., fluocinolone acetonide), fluocinonide, halcinonide, and triamcinolone (e.g., triamcinolone acetonide); Group C (betamethasone type), including beclomethasone, betamethasone, dexamethasone, fluocortolone, halometasone, and mometasone; and Group D (esters), including Group Di halogenated esters (alclometasone dipropionate, betamethasone dipropionate, betamethasone valerate, clobetasol propionate, clobetasone butyrate, fluprednidene acetate, and mometasone furoate), and Group D₂ labile prodrug esters (ciclesonide, cortisone acetate, hydrocortisone aceponate, hydrocortisone acetate, hydrocortisone buteprate, hydrocortisone butyrate, hydrocortisone valerate, prednicarbate, and tixocortol pivalate). In some embodiments, the corticosteroid is selected from hydrocortisone (e.g., hydrocortisone acetate, hydrocortisone aceponate, hydrocortisone buteprate, hydrocortisone butyrate, hydrocortisone cypionate, or hydrocortisone succinate), dexamethasone dexamethasone phosphate), beclometasone (e.g., beclometasone dipropionate), ciclesonide, clobetasol (e.g., clobetasol propionate or clobetasol valerate), clobetasone clobetasone butyrate), desonide, desoxymethasone, desoxycorticosterone desoxycorticosterone acetate), dichlorisone, diflorasone (e.g., diflorasone diacetate), diflucortolone (e.g., diflucortolone valerate), fluadrenolone, fluclarolone fluclarolone acetonide), fludrocortisone (e.g., fludrocortisone acetate), flumethasone (e.g., flumethasone pivalate), fluocinolone (e.g., fluocinolone acetonide), fluocinonide, flucortine (e.g., flucortine butylester), fluocortolone, fluprednidene (e.g., fluprednideneacetate), flurandrenolone, halcinonide, haloinetasone, inethylprednisolone (e.g., methylprednisolone succinate), triamcinolone triamcinolone acetonide), cortisone (e.g., cortisone acetate), cortodoxone, flucetonide, fluradrenalone (e.g., fluradrenalone acetonide), medrysone, alclometasone (e.g., alclometasone dipropionate), amciafel, amcinafide, amcinonide, betamethasone betamethasone dipropionate or betamethasone valerae), budesonide, chlorprednisone (e.g., chlorprednisone acetate), clocortelone, clescinolone, difluprednate, flucloronide, flunisolide, fluoromethalone, fluperolone, fluprednisolone, hydrocortatnate, meprednisone, mometasone mometasone furoate), paramethasone, prednisolone, prednisone, prednicarbate, and tixocortol (e.g., tixocortol pivalate). In some embodiments, the corticosteroid is selected from dexamethasone dexamethasone phosphate), methylprednisolone (e.g., methylprednisolone succinate), and hydrocortisone (e.g., hydrocortisone succinate).

In some embodiments, the composite particles comprise the corticosteroid or salt or ester thereof in an amount of about 70 wt % to about 99 wt %, or about 80 wt % to about 95 wt %. For example, in some embodiments the composite particles comprise the bile acid or salt or ester thereof in an amount of about 70 wt %, 71 wt %, 72 wt %, 73 wt %, 74 wt %, 75 wt %, 76 wt %, 77 wt %, 78 wt %, 79 wt %, 80 wt %. 81 wt %, 82 wt %, 83 wt %, 84 wt %, 85 w′t©, 86 wt %, 87 wt %, 88 wt %, 89 wt %, 90 wt %, 91 wt %, 92 wt %, 93 wt %, wt %, 95 wt %, 96 wt %. 97 wt %, 98 wt %, or 99 wt %, or any range therebetween.

The compound having a steroid core structure can be in the form of a salt. For example, the compound having a steroid core structure can have one or more acidic moieties (e.g., carboxylates), which can form a salt with a suitable cation, such as an alkali metal cation (e.g., sodium, lithium, potassium), an ammonium cation (e.g., NR₄⁺, where each R is independently selected from hydrogen and an alkyl group), or the like. In some embodiments, the salt is a sodium salt. The compound having a steroid core structure also includes esters of steroid compounds. In such ester compounds, one or more hydroxy groups can be functionalized with an acyl group to form an ester. Exemplary ester groups include acetate, adamantoate, benzoate, buteprate, butyrate, caproate, cypionate, enanthate, etabonate, furoate, hexanoate, linoleate, palmitate, pivalate, propionate, tebutate, succinate, undecanoate, undecylenate, valerate, and the like. The compound can also be in the form of a cyclic ketal, such as a cyclic acetal. Compounds with a steroid core structure often have two adjacent hydroxy groups, which can form a cyclic acetal to form an acetonide (e.g., with acetone, particularly at the C16 and C17 positions).

In addition to the compound having a steroid core structure (or salt or ester thereof), in some embodiments, the particles further comprise transition metal ions. In some embodiments, the particles comprise gold, silver, copper, platinum, palladium, nickel, or iron ions. In some embodiments, the particles comprise gold, silver, copper, or iron ions. In some embodiments, the particles comprise gold(III) ions, silver(I) ions, copper(II) ions, nickel(II) ions, palladium(II) ions, platinum(II) ions, iron(II) ions, or iron(III) ions. In some embodiments, the particles comprise gold ions (e.g., Au(III) ions) or iron ions (e.g., Fe(II) ions).

The transition metal ions may be present in the particles in an amount of about 1 wt % to about 30 wt %, or about 5 wt % to about 20 wt %. For example, in some embodiments the composite particles comprise the transition metal ions in an amount of about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, or about 30 wt %, or any range there between.

In some embodiments, in addition to the compound having a steroid cores structure (or salt or ester thereof), the particles further comprise an acid. The acid can be an inorganic acid or an organic acid. Examples of suitable inorganic acids include, but are not limited to: hydrochloric, hydrobromic, hydroiodic, sulfuric, sulfurous, nitric, nitrous, phosphoric, and phosphorous. Examples of suitable organic acids include, but are not limited to: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, hydroxymaleic, hydroxynaphthalene carboxylic, isethionic, lactic, lactobionic, lauric, maleic, rnalic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, tetrafluoroboric, toluenesulfonic, trifluoroacetic, trilluoromethanesulfonic, and valeric. In some embodiments, the acid is hydrochloric acid. In some embodiments, the acid is salicylic acid.

In some embodiments, the particles disclosed herein are essentially free of transition metal nanoparticles (i.e., transition metal nanoparticles in Which the transition metal is in the zero oxidation state (i.e., not a salt), e.g., gold nanoparticles). This feature distinguishes the particles disclosed herein from previously-described composite particles comprising a compound having a steroid core structure, such as those disclosed in International Patent Publication No. WO 2021/011753, which are formed using a double emulsion method. As used herein, the phrase “essentially free of” transition metal nanoparticles means that the nanoparticles include less than 0.5 wt % transition metal nanoparticles, or less than 0.2 wt %, or less than 0.1 wt %, or 0 wt % transition metal nanoparticles. In some embodiments, the particles do not include any detectable amount of metal nanoparticles.

The composite particles can be prepared by a variety of methods. For example, in a first method for making a plurality of composite particles, the method comprises:
(a) providing a first solution comprising a transition metal salt in ethyl acetate;
(b) mixing the first solution with water to form a first emulsion; and
(c) mixing the first emulsion with a second solution, wherein the second solution comprises a compound having a steroid core structure or a salt or ester thereof, to form a final mixture and thereby form the composite particles.

In a second method for making a plurality of composite particles, the method. comprises:
(a) providing a first solution comprising a compound having a steroid core structure or a salt or ester thereof in ethyl acetate;
(b) mixing the first solution with a transition metal salt to form a second solution;
(c) mixing the second solution with water to form a first emulsion;
(d) mixing the first emulsion with a solution of a compound having a steroid core structure or a salt or ester thereof in water to form a final mixture and thereby form the composite particles.

In a third method for making a plurality of composite particles, the method comprises:
(a) providing a first solution comprising a transition metal salt in water;
(b) mixing the first solution with ethyl acetate to form a first emulsion;
(c) mixing the first emulsion with a solution of a compound having a steroid core structure or a salt or ester thereof in water to form a final mixture and thereby form the composite particles.

In a fourth method for making a plurality of composite particles, the method comprises:
(a) providing a first solution comprising an acid, a compound having a steroid core structure or a salt or ester thereof, and water;
(b) mixing the first solution with ethyl acetate to form a first emulsion;
(c) mixing the first emulsion with a solution of an acid in water to form a final mixture and thereby form the composite particles.

In a fifth method for making a plurality of composite particles, the method comprises:
(a) providing a first solution comprising an acid in ethyl acetate;
(b) mixing the first solution with a second solution comprising a compound having a steroid core structure or a salt or ester thereof and water, to form a first emulsion,
(c) mixing the first emulsion with a third solution comprising a compound having a steroid core structure or a salt or ester thereof and water, to form a final mixture and thereby form the composite particles.

In some embodiments of the first, second, and third methods, the transition metal salt is a gold(III) salt, a silver(I) salt, a copper(II) salt, a nickel(II) salt, a palladium(II) salt, a platinum(II) salt, an iron(II) salt, or an iron(III) salt. In some embodiments, the transition metal salt is a gold(III) salt. In some embodiments, the transition metal salt is gold(III) chloride. In some embodiments, the transition metal salt is an iron(II) salt. In some embodiments, the transition metal salt is iron(II) sulfate. In some embodiments, the transition metal salt is a silver(I) salt. In some embodiments, the transition metal salt is silver(I) nitrate. In some embodiments, the transition metal salt is a copper(II) salt. In some embodiments, the transition metal salt is copper(II) chloride.

In some embodiments of the first, second, third, fourth, and fifth methods, the compound having a steroid core structure is a bile acid or a salt or ester thereof. In some embodiments, the bile acid is selected from cholic acid, deoxycholic acid, chenodeoxycholic acid, lithocholic acid, glycocholic acid, taurocholic acid, glycodeoxycholic acid, taurodeoxycholic acid, glycochenodeoxycholic acid, taurochenodeoxycholic acid, glycolithocholic acid, taurolithocholic acid, ursodeoxycholic acid, glycoursodeoxycholic acid, tauroursodeoxycholic acid, and obeticholic acid. In some embodiments, the bile acid is selected from cholic acid, deoxycholic acid, ursodeoxycholic acid, and chenodeoxycholic acid. In some embodiments, the compound having a steroid core structure is a bile acid salt. In some embodiments, the bile acid salt is a sodium or potassium salt. In some embodiments, the bile acid salt is a sodium salt. Accordingly, in some embodiments, the bile acid salt is selected from sodium cholate, sodium deoxycholate, sodium ursodeoxycholate, and sodium chenodeoxycholate. In some embodiments, the bile acid salt is selected from sodium cholate and sodium deoxycholate. In some embodiments, the bile acid salt is sodium cholate. In some embodiments, the bile acid salt is sodium deoxycholate. In some embodiments, the bile acid salt is sodium ursodeoxycholate. In some embodiments, the bile acid salt is sodium chenodeoxycholate.

In some embodiments of the first, second, third, fourth, and fifth methods, the compound having a steroid core structure is a corticosteroid compound or a salt or ester thereof. In some embodiments, the corticosteroid compound is selected from dexamethasone, methylprednisolone, and hydrocortisone, or an ester thereof. In some embodiments, the corticosteroid compound is dexamethasone or an ester thereof. In some embodiments, the corticosteroid compound is methylprednisolone or an ester thereof. In some embodiments, the corticosteroid compound is hydrocortisone or an ester thereof.

In some embodiments, of the first, second, third, fourth, and fifth methods, the composite particles form immediately upon formation of the final mixture. In other embodiments, the first, second, and third methods further comprise a step of stirring a final mixture. In some embodiments, the final mixture is stirred for about 1 second to about 15 minutes, during which time the composite particles form. For example, the stirring step may comprise stirring the final mixture for about 5 seconds, about 10 seconds, 15 seconds, about 30 seconds, about 45 seconds, about 60 seconds, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, or about 15 minutes.

The first, second, third, fourth, and fifth methods may further comprise an additional step of separating the composite particles from the mixture. The separating step can include filtering using a filter having an appropriate pore size. The separating step may alternatively or additionally include centrifugation. For example, centrifugation (e.g., at 100-5000 rpm, such as 100, 200, 300, 400, 500, 600, 700, 800, 900, 1000, 1500, 2000, 2500, 3000, 3500, 4000, 4500, or 5000 rpm) may effectively separate the composite particles from other components. The composite particles may be recovered in the pellet from the centrifugation.

In some embodiments of the first, second, third, fourth, and fifth methods, the method does not comprise a heating step. Compared to other methods of preparing composite particles that include metal nanoparticles, which involve use of a reducing agent to form transition metal nanoparticles and require a heating step to reduce the transition metal ions to their elemental state, the methods disclosed herein do not require a reduction step, and accordingly no heating step is required. In some embodiments of the first, second, and third methods, the methods are conducted at ambient temperature.

Similarly, in the first, second, third, fourth, and fifth methods, the compositions do not include a reducing agent (e.g., sodium citrate or sodium ascorbate). A reducing agent is not necessary as the composite particles form without being templated by the formation of transition metal nanoparticles.

In some embodiments, the method further comprises removing the solvent from the final mixture after the incubation step. The solvent can be removed using a variety of methods, such as evaporation at ambient temperature and pressure, or evaporation with heating, or evaporation under reduced pressure. In some embodiments, the solvent is removed by evaporation at ambient temperature and pressure.

Specific methods of making composite particles disclosed herein are described in the Examples. In some embodiments, provided herein are particles produced by any of the above described methods.

In some embodiments, the composite particles consist essentially of a compound having a steroid core structure, or the salt or ester thereof, and transition metal ions. In some embodiments, the particle consists essentially of a bile acid or salt or ester thereof and gold, silver, copper, platinum, palladium, nickel, or iron ions. In some embodiments, the composite particles consist essentially of a bile acid or a salt or ester thereof, and gold, silver, copper, or iron ions. in some embodiments, the composite particles consist essentially of a bile acid or a salt or ester thereof, and gold ions (e.g., Au(III) ions) or iron ions (e.g., iron(II) ions). In some embodiments, the composite particles consist essentially of a corticosteroid, or a salt or ester thereof, and transition metal ions. In some embodiments, the particle consists essentially of a corticosteroid, or a salt or ester thereof, and gold, silver, copper, platinum, palladium, nickel, or iron ions. In some embodiments, the composite particles consist essentially of a corticosteroid, or a salt or ester thereof, and gold, silver, copper, or iron ions. In some embodiments, the composite particles consist essentially of a corticosteroid, or a salt or ester thereof, and gold ions (e.g., Au(III) ions) or iron ions (e.g., iron(II) ions). In such embodiments, the composite particles do not include, or are substantially free of, other components such as small molecules, polymers, and the like.

In some embodiments, the composite particles consist essentially of a compound haying a steroid core structure, or the salt or ester thereof, and an acid. In some embodiments, the particle consists essentially of a bile acid or salt or ester thereof and an acid selected from hydrochloric acid and salicylic acid. In some embodiments, the composite particles consist essentially of a corticosteroid, or a salt or ester thereof, and an acid. In some embodiments, the particle consists essentially of a corticosteroid, or a salt or ester thereof, an acid selected from hydrochloric acid and salicylic acid. In such embodiments, the composite particles do not include, or are substantially free of, other components such as metal nanoparticles, metal ions, small molecules, polymers, and the like.

In some embodiments, the composite particles do not include a polymer. For example, the composite particles do not include polymers such as polyesters, including polylactic-co-glycolic acid) (PLGA), or anionic polymers such as polysaccharides (e.g., dextran sulfate, heparin, heparin sulfate, chondroitin sulfate, hyaluronic acid, or alginic acid), nucleic acid polymers, and the like. For example, in the composite particles of the disclosure, the compound having the steroid core structure (e.g., the bile acid or the corticosteroid) is not conjugated to a polymer. in some embodiments, the composite particles do not include phosphatidylcholine.

In other embodiments, the particles may further comprise other components. For example, in some embodiments, the particles further include a targeting ligand. Targeting ligands are well-known to those skilled in the art; exemplary targeting ligands are described by Srinivasarao et al. “Ligand-Targeted Drug Delivery,” Chem. Rev. 2017, 117(19), 12133-12164, which is incorporated herein by reference. Exemplary targeting ligands include antibodies to molecules that are expressed on cell surfaces; for example, certain lipoma cells are known to overexpress CD34, and an anti-CD34 antibody could be used as a targeting ligand for those cells.

In other embodiments, the particles further include an additional therapeutic agent. Any suitable therapeutic agent can be used. Exemplary therapeutic agents include those described in Harrison's Principles of internal Medicine, 20th Edition, Eds. J. L. Jameson et al., McGraw-Hill Education (2018); Physicians' Desk Reference, 71st Edition, PDR Network (2017); and Goodman & Gilman's The Pharmacological Basis of Therapeutics, 13^{th} Edition, Eds. L. L. Brunton et al., 2017; United States Pharmacopeia—The National Formulary, USP 42-NF 37, 2019; the contents of each of which are incorporated herein by reference.

The composite particles can be characterized by a wide variety of techniques. For example, particles can be imaged using scanning electron microscopy (SEM) to determine their size and shape. The elemental composition can be confirmed using elemental analysis, for example using energy-dispersive spectroscopy (EDS) and/or X-ray photoelectron spectroscopy (XPS). The presence of the bile acid can further be confirmed using techniques such as high-performance liquid chromatography (HPLC), Fourier-transform infrared (FTIR) spectroscopy, and nuclear magnetic resonance (NMR) spectroscopy.

In some embodiments, the composite particles have a hexagonal prism shape. Such particles can be characterized by their diagonal length, i.e. the length between two opposite vertices of the hexagon, as well as their height. In some embodiments, the composite particles have a hexagonal prism shape with an average diagonal length of about 2.5 μm to about 10 μm, or about 3.0 μm to about 9.0 μm (e.g., about 2.5, 3.0, 3.5, 4.0, 4.5, 5.0, 5.5, 6.0, 6.5, 7.0, 7.5, 8.0, 8.5, 9.0, 9.5, or 10 μm, or any range therebetween). In some embodiments, the composite particles have a hexagonal prism shape with an average height of about 2.5 μm to about 6.5 μm (e.g., about 2.5, 3.0, 3.5, 4.0, 4.5, 5.0, 5.5, 6.0, or 6.5 μm, or any range therebetween).

In some embodiments, the composite particles have a rod shape. In some embodiments, the composite particles have a rod shape with an average length of about 2.5 μm to about 100 μm, or about 10 μm to about 50 μm (e.g., about 2.5, 5.0, 10, 15, 20, 25, 30, 35, 40, 45, 50, 55, 60, 65, 70, 75, 80, 85, 90, 95, or 100 μm, or any range therebetween).

In some embodiments, the composite particles have a hexagonal sheet shape. Such particles can be characterized by the length of the long and short sides of the sheet. In some embodiments, the composite particles have a hexagonal sheet shape with an average long side length of about 10 μm to about 50 μm (e.g., about 10, 15, 20, 25, 30, 35, 40, 45, or 50 μm, or any range therebetween), and a short side length of 5 μm to 20 μm (e.g., about 5, 7.5, 10, 12.5, 15, 17.5, or 20 μm, or any range therebetween).

In some embodiments, the composite particles have a spherical shape. In some embodiments, the sphere has a diameter of 1 μm to 10 μm (e.g., about 1 μm, about 2 μm, about 3 μm, about 4 μm, about 5 μm, about 6 μm, about 7 μm, about 8 μm, about 9 μm, or about 10 μm, or any range therebetween).

### Compositions

In another aspect, the disclosure provides a composition comprising a plurality of the composite particles described herein. In some embodiments, the compositions further comprise a pharmaceutically acceptable carrier.

As used herein, the term “pharmaceutically acceptable carrier” refers to a pharmaceutically-acceptable material, composition or vehicle for administration of an active agent described herein. Pharmaceutically acceptable carriers include any and all solvents, dispersion media, coatings, antibacterial and antifungal agents, isotonic and absorption delaying agents, and the like which are compatible with the activity of the bile acid or salt or ester thereof and are physiologically acceptable to the subject. Some examples of materials that can serve as pharmaceutically-acceptable carriers include: (i) sugars, such as lactose, glucose and sucrose; (ii) starches, such as corn starch and potato starch (iii) cellulose, and its derivatives, such as sodium carboxymethyl cellulose, methylcellulose, ethyl cellulose, microcrystalline cellulose and. cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, corn oil and soybean oil; (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C2-C12 alcohols, such as ethanol; and (xxv) other non-toxic compatible substances employed in pharmaceutical formulations. Wetting agents, coloring agents, release agents, coating agents, sweetening agents, flavoring agents, perfuming agents, preservative and antioxidants can also be present in the formulation.

For formulations described herein to be administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Pharmaceutically acceptable carriers can vary in a formulation described herein, depending on the administration route. The formulations described herein can be delivered via any administration mode known to a skilled practitioner. For example, the formulations described herein can be delivered in a systemic manner, via administration routes such as, but not limited to, oral and parenteral, including intravenous, intramuscular, intraperitoneal, intradermal, and subcutaneous. in some embodiments, the formulations described herein are in a form that is suitable for injection, particularly subcutaneous injection. In other embodiments, the formulations described herein are formulated for oral administration, such as a tablet or a capsule.

When administering parenterally, a formulation described herein can be generally formulated in a unit dosage injectable form (solution, suspension, emulsion). The formulations suitable for injection include sterile aqueous solutions or dispersions. The carrier can be a solvent or dispersing medium containing, for example, water, buffers (e.g., phosphate buffered saline), polyol (for example, glycerol, propylene glycol, liquid polyethylene glycol, and the like), salts (e.g., sodium chloride), or suitable mixtures thereof. In some embodiments, the pharmaceutical carrier can be a saline solution. In some embodiments, the pharmaceutical carrier can be a buffered solution (e.g., PBS). Where necessary, the composition may also include a solubilizing agent and a local anesthetic such as lignocaine to ease pain at the site of the injection.

The formulations can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE,” 17th edition, 1985, incorporated herein by reference, may be consulted to prepare suitable preparations, without undue experimentation. With respect to formulations described herein, however, any vehicle, diluent, or additive used should have to be biocompatible with the active agents described herein. Those skilled in the art will recognize that the components of the formulations should be selected to be biocompatible with respect to the active agent. This will present no problem to those skilled in chemical and pharmaceutical principles, or problems can be readily avoided by reference to standard texts or by simple experiments (not involving undue experimentation).

For in vivo administration, the formulations described herein can be administered with a delivery device, e.g., a syringe. Accordingly, an additional aspect described herein provides for delivery devices comprising at least one chamber with an outlet, wherein the at least one chamber comprises a pre-determined amount of any formulation described herein and the outlet provides an exit for the formulation enclosed inside the chamber. In some embodiments, a delivery device described herein can further comprise an actuator to control release of the formulation through the outlet. Such delivery device can be any device to facilitate the administration of any formulation described herein to a subject, e.g., a syringe, a dry powder injector, a nasal spray, a nebulizer, or an implant such as a microchip, e.g., for sustained-release or controlled release of any formulation described herein.

In some embodiments, the compositions do not include phosphatidylcholine.

### Methods of Use

The composite particles can be used in a variety of methods, such as a methods of treating a disorder in a subject.

As used herein, the term “subject” includes human and non-human animals. Exemplary human subjects include a human patient having a disorder, e.g., a disorder described herein, or a normal subject. The term “non-human animals” includes all vertebrates, e.g. , non-mammals (such as chickens, amphibians, reptiles) and mammals, such as non-human primates, domesticated andlor agriculturally useful animals, e.g., horse, sheep, dog, cat, cow, pig, etc.

As used herein, the term “treat” or “treating” a subject having a disorder refers to subjecting the subject to a regimen, e.g., the administration of a particle or a composition described herein, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

Bile acids and their salts have been proposed as therapeutic agents for treatment of different conditions including bile acid synthesis disorders and peroxisomal disorders (see, e.g., Klouwer et al. Orphanet J. Rare Dis., 2015, 10, 151; W. T. Elliot. Internal Medicine Alert, 2015, 37), primary biliary cholangitis (also known as primary biliary cirrhosis; see, e.g., Hirschfield et al. Gut, 2018, 67, 1568), primary sclerosing cholangitis (see, e.g., Mikov et al. Eur. J. Drug Metab. Pharmacokinet., 2006, 31, 237), cardiometabolic diseases (see, e.g., Ikemoto et al. Am. J. Physiol. Metab., 1997, 273, E37), gallstones and bile duct stones (see, e.g., Fini et al. J. Pharm. Sci. 85(9), 971); Lansford et al. Gut, 1974, 15, 48), non-alcoholic fatty liver disease (see, e.g., Quintero et al. J. Physiol. Biochem., 2014, 70, 667; Gabbi et al. Dig. Liver Dis., 2012, 44, 1018), type-2 diabetes (see, e.g., Gabbi 2012), human immunodeficiency virus type 1 (HIV-1) (see, e.g., Mikov 2006), acute pancreatitis (see, e.g., Mikov 2006), cancer (see, e.g., Goossens et al. Pharmacol. Ther. 203, 107396 (2019); Zeng et al. Nutr. Cancer 62, 85-92 (2009); Pardi et al. Gastroenterology 124, 889-893 (2003); Milovic et al. Eur. J. Clin. Invest. 32, 29-34 (2002); Schlottmann et al. Cancer Res. 60, 4270-4276 (2000); Šarenac et al. Front. Pharmacol. 10:484 (2019), doi: 10.3389/fphar.2019.00484), and removal of undesired fat (Yagima Odo et al. Dermatologic Surg. 33, 178-189 (2007)). Bile acids and salts thereof are also useful for the non-surgical removal of a localized fat deposit in a subject. For example, deoxycholic acid injections have been approved by the U.S. FDA for improving the appearance of moderate to severe convexity or fullness associated with submental fat in adults. However, many injections are needed, with a single treatment including up to 50 injections, and up to six single treatments may be needed for effective treatment. Accordingly, it is contemplated that the composite particles disclosed herein provide controlled release of the bile acid or salt or ester thereof, which reduces the number of injections required for effective treatment.

Corticosteroids are used to treat a variety of conditions, including endocrine disorders (e.g., primary or secondary adrenocortical insufficiency, congenital adrenal hyperplasia, nonsuppurative thyroiditis, or hypercalcemia associated with cancer), rheumatic disorders (e.g., rheumatoid arthritis (including juvenile rheumatoid arthritis), ankylosing spondylitis, acute and subacute bursitis, synovitis of osteoarthritis, acute nonspecific tenosynovitis, post-traumatic osteoarthritis, psoriatic arthritis, epicondylitis, or acute gouty arthritis), collagen diseases (e.g., during an exacerbation or as maintenance therapy in systemic lupus erythematosus, systemic dertnatotnyositis (polymyositis), or acute rheumatic carditis), derinatologic diseases (e.g., bullous dermatitis herpetiformis, severe erythema multiforme (Stevens-Johnson syndrome), severe seborrheic dermatitis, exfoliative dermatitis, mycosis fungoides, pemphigus, or severe psoriasis), allergic states (e.g., seasonal or perennial allergic rhinitis, drug hypersensitivity reactions, serum sickness, contact dermatitis, bronchial asthma, or atopic dermatitis), ophthalmic diseases (e.g., severe acute and chronic allergic and inflammatory processes involving the eye and its adnexa, such as allergic corneal marginal ulcers, herpes zoster ophthalmicus, anterior segment inflammation, diffuse posterior uveitis and choroiditis, sympathetic ophthalmia, keratitis, optic neuritis, allergic conjunctivitis, chorioretinitis, uveitis and ocular inflammatory conditions unresponsive to topical steroids, or iritis and iridocyclitis), respiratory diseases (e.g., symptomatic sarcoidosis, berylliosis, Loeffler's syndrome, fulminating or disseminated pulmonary tuberculosis, aspiration pneumonitis, chronic obstructive pulmonary disease (COPD), allergic bronchopulmonary aspergillosis, asthma, hypersensitivity pneumonitis, idiopathic bronchiolitis obliterans with organizing pneumonia, idiopathic eosinophilic pneumonias, idiopathic pulmonary fibrosis, or pneumocystis carinii pneumonia), hematologic disorders (e.g., idiopathic thrombocytopenic purpura, secondary thrombocytopenia, acquired (autoimmune) hemolytic anemia, erythroblastopenia (RBC anemia), congenital (erythroid) hypoplastic anemia, Diamond-Blackfan anemia, or pure red cell aplasia), neoplastic diseases (e.g., leukemias and lymphomas in adults, or acute leukemia of childhood), gastrointestinal diseases (e.g., during acute episodes of ulcerative colitis, regional enteritis, or Crohn's disease), nervous system disorders (e.g., acute exacerbations of multiple sclerosis), inflammatory disorders, renal diseases (e.g., to induce a diuresis or remission of proteinuria in nephrotic syndrome of the idiopathic type or that is due to lupus erythematosus), and other conditions (e.g., tuberculous meningitis with subarachnoid block or impending block when used concurrently with appropriate antituberculous chemotherapy, trichinosis with neurological or myocardial involvement). Corticosteroids are often used for treatment of severe allergies or skin problems, asthma, and arthritis.

Other compounds having steroid core structures are also used in treating a wide variety of disorders. For example, listed below are compounds having steroid core structures, and disorders they are used to treat in humans. in some embodiments, the composite particles described herein comprise the indicated compounds, and the disclosure provides a method of using such particles to treat the indicated disorders and/or for the following purposes:

Testosterone (Testosterone, testosterone enanthate, and testosterone cypionate forms): Breast cancer, metastatic; delayed puberty; Hypogonadism, hypogonadotropic (congenital or acquired); Hypogonadism, primary (congenital or acquired); Hormone therapy for transgender males (female-to-male);

Testosterone undecanoate: Breast cancer, metastatic; Delayed puberty; Hypogonadism, hypogonadotropic (congenital or acquired); Ilypogonadistn, primary (congenital or acquired); Hormone therapy for transgender males (female-to-male);

Exemestane: Breast cancer; First-line adjuvant treatment of estrogen receptor-positive early breast cancer in postmenopausal women; Risk reduction for invasive breast cancer in postmenopausal women;

Formestane: Treatment of advanced breast cancer in postmenopausal women;

Mesterolone: Androgen deficiency; hypogonadism; infertility; delayed puberty;

Fluoxymesterone: Breast cancer, metastatic (females); Delayed puberty (males); Hypogonadism, primary or hypogonadotrophic (males);

Methyltestosterone: Breast cancer, metastatic (females); Delayed puberty (males); Hypogonadism, primary or hypogonadotropic (males);

Oxandrolone: Weight gain (adjunctive therapy); Burns, severe (adjunctive therapy);

Oxymetholone: Anemia; Fanconi anemia;

Mestranol (administered with norethindrone): Abnormal uterine bleeding; Dysmenorrhea; Menstrual bleeding menorrhagia); Pain associated with endometriosis; Polycystic ovary syndrome (PCOS) in women with menstrual irregularities and hirsutism/acne;

Norethindrone: Abnormal uterine bleeding; Amenorrhea, secondary; Contraception; Endometriosis;

Danazol Endometriosis; Hereditary angioedema (HAE), prophylaxis; Cyclic breast pain (mastalgia) associated with benign breast disorders; Immune thrombocytopenia, refractory;

Gestrinone: Endometriosis;

Levonorgestrel: Contraception; Heavy menstrual bleeding; Endometrial hyperplasia;

Lynestrenol: Prevention of pregnancy; treatment of polymenorrhea, menorrhagia, metrorrhagia, primary and secondary amenorrhea or oligomenorrhea; treatment of benign breast disease; treatment of endometrial carcinoma; adjunct to estrogen therapy in peri- and post-menopausal women to prevent endometrial hyperplasia; treatment of endometriosis; suppression of ovulation, ovulation pain or menstruation, or dysmenorrhea; to postpone the onset of menstruation;

Norgestrel (administered with ethinyl estradiol): Contraception; Abnormal uterine bleeding; Dysmenorrhea; Hirsutism; Menstrual bleeding (menorrhagia); Pain associated with endometriosis; Polycystic ovary syndrome (PCOS) in women with menstrual irregularities and hirsutistn/acne;

Desogestrel: Contraception; Abnormal uterine bleeding; Dystnenorrhea; Hirsutism; Menstrual bleeding (menorrhagia); Polycystic ovary syndrome (PCOS) in women with menstrual irregularities and hirsutism/acne;

Etonogestrel: Contraception;

Tibolone: Treatment of symptoms associated with menopause; prevention of postmenopausal osteoporosis in high-risk women with contraindications or an intolerance to first-line therapy;

Ethynodiol diacetate (administered with ethinyl estradiol): Contraception; Abnormal uterine bleeding; Dysmenorrhea; Hirsutism; Menstrual bleeding (menorrhagia); Pain associated with endometriosis; Polycystic ovary syndrome (PCOS) in women with menstrual irregularities and hirsutismlacne;

Cyproterone: Prostate cancer; Hormone therapy for transgender females (male-to-female); Paraphilia;

Megestrol acetate: Anorexia or cachexia; Breast cancer; Endometrial cancer; Treatment of cancer-related cachexia;

Abiraterone acetate: Prostate cancer, metastatic;

Dienogest: Endometriosis;

Mifepristone: To control hyperglycemia occurring secondary to hypercortisolism in adult patients with endogenous Cushing syndrome who have type 2 diabetes mellitus or glucose intolerance and who failed surgery or who are not surgical candidates; Medical termination of intrauterine pregnancy through 70 days gestation, in combination with misoprostol; Early pregnancy loss;

Drospirenone: Contraception;

Spironolactone: Ascites due to cirrhosis; Heart failure with reduced ejection fraction; Hypertension; Primary hyperaldosteronism; Acne vulgaris, females; Heart failure with preserved ejection fraction; Heart failure with reduced ejection fraction; Hirsutism, females; Hormone therapy for transgender females, male-to-female; Post myocardial infarction, complicated by reduced ejection fraction;

Estradiol: Breast cancer, metastatic; Hypoestrogenism (female); Osteoporosis prevention (female); Prostate cancer, advanced; Vasomotor symptoms associated with menopause; Vulvar and vaginal atrophy associated with menopause; Functional hypothalamic amenorrhea with low bone density (young adult females); Hormone therapy for transgender females (male-to-female);

Polyestradiol phosphate: Palliative treatment of advanced, inoperable carcinoma of the prostate;

Megestrol: Anorexia or cachexia; Breast cancer; Endometrial cancer; Treatment of cancer-related cachexia;

Estramustine: Prostate cancer (metastatic castration-resistant);

Estramustine phosphate: Prostate Cancer;

Estrone: Vulvar and vaginal atrophy;

Estropipate: Hypoestrogenism, female; Osteoporosis prevention; Vasomotor symptoms due to menopause; Vulvar and vaginal atrophy due to menopause;

Progesterone: Prevention of endometrial hyperplasia in nonhysterectomized, postmenopausal women who are receiving conjugated estrogens; treatment of secondary amenorrhea; Treatment of amenorrhea or abnormal uterine bleeding due to hormonal imbalance in the absence of organic pathology, such as submucous fibroids or uterine cancer; Part of assisted reproductive technology (ART) for infertile women with progesterone deficiency; To support embryo implantation and early pregnancy by supplementation of corpus luteal function as part of ART for infertile women; Reduce the risk of recurrent spontaneous preterm birth;

Dydrogesterone: Treatment of various conditions caused by progesterone deficiencies;

Hydroxyprogesterone caproate: To reduce the risk of preterm birth in women with a singleton pregnancy who have a history of singleton spontaneous preterm birth; Treatment of advanced (stage III or IV) uterine adenocarcinoma; management of amenorrhea (primary and secondary) and abnormal uterine bleeding due to hormonal imbalance in the absence of organic pathology (e.g., submucous fibroids or uterine cancer); as a test for endogenous estrogen production; production of secretory endometrium and desquatnation;

Medroxyprogesterone acetate: Abnormal uterine bleeding; Amenorrhea, secondary; Contraception; Endometrial hyperplasia prevention; Endometrial carcinoma; Endometriosis; Abnormal uterine bleeding, acute; Endometrial hyperplasia; Hot flashes; Paraphilia/hypersexuality;

Segesterone acetate (administered with ethinyl estradiol): Contraceptive;

Norelgestromin (administered with ethinyl estradiol): Contraception; Polycystic ovary syndrome (PCOS) in women with menstrual irregularities and hirsutism/acne;

Norgestimate (administered with estradiol): Osteoporosis prevention; Vasomotor symptoms associated with menopause; Vulvar and vaginal atrophy associated with menopause;

Norgestimate (administered with ethinyl estradiol): Acne vulgaris; Contraception; Abnormal uterine bleeding; Dysmenorrhea; Hirsutism; Menstrual bleeding (menorrhagia); Polycystic ovary syndrome (PCOS) in women with menstrual irregularities and hirsutism/acne;

Cortisol: Allergic states: Control of severe or incapacitating allergic conditions intractable to adequate trials of conventional treatment in drug hypersensitivity reactions, perennial or seasonal allergic rhinitis, serum sickness, transfusion reactions, or acute noninfectious laryngeal edema (epinephrine is the drug of first choice); Dermatologic diseases: Atopic dermatitis; bullous dermatitis herpetiformis; contact dermatitis; exfoliative dermatitis; exfoliative erythroderma; pemphigus; severe erythema multiforme (Stevens-Johnson syndrome); severe psoriasis; severe seborrheic dermatitis; mycosis fungoides; Edematous slaws: To induce diuresis or remission of proteinuria in the nephrotic syndrome, without uremia, of the idiopathic type or that due to lupus erythematosus; Endocrine disorders: Acute adrenocortical insufficiency; congenital adrenal hyperplasia; hypercalcemia associated with cancer; nonsuppurative thyroiditis; primary or secondary adrenocortical insufficiency; preoperatively and in the event of serious trauma or illness, in patients with known adrenal insufficiency or when adrenocortical reserve is doubtful; shock unresponsive to conventional therapy if adrenocortical insufficiency exists or is suspected; GI diseases: To tide the patient over a critical period of the disease in ulcerative colitis and regional enteritis; Hematologic disorders: Acquired (autoimmune) hemolytic anemia; congenital (erythroid) hypoplastic anemia (Diamond Blackfan anemia); erythroblastopenia (RBC anemia); immune thrombocytopenia (formerly known as idiopathic thrombocytopenic purpura) in adults; pure red cell aplasia; select cases of secondary thrombocytopenia; Neoplastic diseases: Palliative management of leukemias and lymphomas (adults); acute leukemia of childhood; Nervous system: Cerebral edema associated with primary or metastatic brain tumor, or craniotomy; Ophthalmic diseases: Severe acute and chronic allergic and inflammatory processes involving the eye, such as allergic conjunctivitis; allergic corneal marginal ulcers; anterior segment inflammation; chorioretinitis; diffuse posterior uveitis and choroiditis; herpes zoster ophthalmicus; iritis and iridocyclitis; keratitis; optic neuritis; sympathetic ophthalmia; other ocular inflammatory conditions unresponsive to topical corticosteroids; Respiratory diseases: Aspiration pneumonitis; bronchial asthma; berylliosis; fulminating or disseminated pulmonary tuberculosis when used concurrently with appropriate antituberculous chemotherapy; idiopathic eosinophilic pneumonias; Loeffler syndrome (not manageable by other means); symptomatic sarcoidosis; Rheumatic disorders: As adjunctive therapy for short-term administration in acute and subacute bursitis, acute gouty arthritis, acute nonspecific tenosynovitis, ankylosing spondylitis, epicondylitis, posttraumatic osteoarthritis, psoriatic arthritis, rheumatoid arthritis, including juvenile rheumatoid arthritis, synovitis of osteoarthritis; during an exacerbation or as maintenance therapy in acute rheumatic carditis, dermatomyositis (polymyositis), temporal arteritis, and systemic lupus erythematosus; Miscellaneous: Trichinosis with neurologic or myocardial involvement; tuberculous meningitis with subarachnoid block or impending block when used concurrently with appropriate antituberculous chemotherapy; In-hospital cardiac arrest; Septic shock; Thyroid storm;

Cortisone (Cortisone and cortisone acetate forms): Allergic states: Control of severe or incapacitating allergic conditions intractable to adequate trials of conventional treatment of atopic dermatitis, bronchial asthma, contact dermatitis, drug hypersensitivity reactions, seasonal or perennial allergic rhinitis, and serum sickness; Dermatologic diseases: Bullous dermatitis herpetiformis, exfoliative dermatitis, mycosis fungoides, pemphigus, severe erythema multiforme (Stevens-Johnson syndrome), severe psoriasis, severe seborrheic dermatitis; Endocrine disorders: Congenital adrenal hyperplasia, hypercalcemia associated with cancer, nonsuppurative thyroiditis, primary or secondary adrenocortical; Gastrointestinal diseases: To tide the patient over a critical period of the disease in regional enteritis and ulcerative colitis; Hematologic disorders: Acquired (autoimmune) hemolytic anemia, congenital (erythroid) hypoplastic anemia, erythroblastopenia (red blood cell [RBC] anemia), immune thrombocytopenia (formerly known as idiopathic thrombocytopenic purpura) in adults, secondary thrombocytopenia in adults; Neoplastic diseases: Palliative management of leukemias and lymphomas in adults; acute leukemia of childhood; Ophthalmic diseases: Severe acute and chronic allergic and inflammatory processes involving the eye and its adnexa (e.g., allergic conjunctivitis, allergic corneal marginal ulcers, anterior segment inflammation, chorioretinitis, diffuse posterior uveitis and choroiditis, keratitis, herpes zoster ophthalmicus, iritis and iridocyclitis, optic neuritis, sympathetic ophthalmia); Renal diseases: To induce diuresis or remission of proteinuria in nephrotic syndrome, without uremia, of the idiopathic type or that is caused by lupus erythematosus; Respiratory diseases: Aspiration pneumonitis, berylliosis, fulminating or disseminated pulmonary tuberculosis when used concurrently with appropriate antituberculosis chemotherapy, Loeffler syndrome not manageable by other means, symptomatic sarcoidosis; Rheumatic disorders: Adjunctive therapy for short-term administration (to tide the patient over an acute episode or exacerbation) in acute and subacute bursitis; acute gouty arthritis; acute nonspecific tenosynovitis; ankylosing spondylitis; epicondylitis; posttraumatic osteoarthritis; psoriatic arthritis; rheumatoid arthritis (RA), including juvenile RA (select cases may require low-dose maintenance therapy); and synovitis of osteoarthritis. During an exacerbation or as maintenance therapy in select cases of acute rheumatic carditis, systemic dermatomyositis (polymyositis), and systemic lupus erythematosus; Miscellaneous: Tuberculous meningitis with subarachnoid block or impending block when used concurrently with appropriate antituberculous chemotherapy; trichinosis with neurologic or myocardial involvement;

Fluorometholone: Ocular inflammation: Treatment of steroid-responsive inflammation of the palpebral and bulbar conjunctiva, cornea, and anterior segment of the eye;

Difluprednate: Inflammationlpain: Treatment of inflammation and pain following ocular surgery; Uveitis: Treatment of endogenous anterior uveitis;

Fludrocortisone (Fludrocortisone acetate form): Adrenal insufficiency, primary; Congenital adrenal hyperplasia, classic; Idiopathic orthostatic hypotension; Septic shock;

Fluocinolone (fluocinolone acetonide form): Body oil: Treatment of moderate to severe atopic dermatitis in pediatric patients ≥3 months; treatment of atopic dermatitis in adults; Cream, ointment, topical solution: Relief of inflammatory and pruritic manifestations of corticosteroid-responsive dermatoses; Scalp oil: Treatment of psoriasis of the scalp in adults; Shampoo: Treatment of seborrheic dermatitis of the scalp; Relief of chronic eczematous external otitis; Diabetic macular edema; Uveitis;

Loteprednol (Loteprednol etabonate form): Ophthalmic inflammatory conditions (0.5% suspension): Treatment of ocular, anterior segment inflammation that is expected to be responsive to topical corticosteroid therapy; Postoperative inflammation/pain (0.38% gel; 0.5% suspension/ointment/gel; 1% suspension): Treatment of postoperative inflammation and pain following ocular surgery; Seasonal allergic conjunctivitis (0.2% suspension): Temporary relief of signs and symptoms of seasonal allergic conjunctivitis;

Methylprednisolone (methylprednisolone acetate and rnethylprednisolone succinate forms): Oral, IM (acetate or succinate), and IV (succinate only) administration: Anti-inflammatory or immunosuppressant agent in the treatment of a variety of diseases, including those of hematologic (e.g., immune thrombocytopenia, warm autoimmune hemolytic anemia), allergic, gastrointestinal (e.g., Crohn disease, ulcerative colitis), inflammatory, neoplastic, neurologic (e.g., multiple sclerosis), rheumatic (e.g., antineutrophil cytoplasmic antibody-associated vasculitis, dermatomyositislpolymyositis, giant-cell arteritis, gout [acute flare], giant cell arteritis, mixed cryoglobulinemia syndrome, polyarteritis nodosa, rheumatoid arthritis, systemic lupus erythematosus), and/or autoimmune origin; intra-articular or soft tissue administration (acetate only): Gout (acute flare), acute and subacute bursitis, acute nonspecific tenosynovitis, epicondylitis, rheumatoid arthritis, and/or synovitis of osteoarthritis; intralesional administration (acetate only): Alopecia areata; discoid lupus erythematosus; keloids; localized hypertrophic, infiltrated, inflammatory lesions of granuloma annulare, lichen planus, lichen simplex chronicus (neurodermatitis), and psoriatic plaques; and necrobiosis lipoidica diaheticorum. May be useful in cystic tumor of an aponeurosis or tendon (ganglia); Acute respiratory distress syndrome, moderate to severe; Cardiac transplant: Antibody-mediated rejection; Chronic obstructive pulmonary disease; Deceased organ donor management; Graft-vs-host disease, acute; In-hospital cardiac arrest; Nausea and vomiting of pregnancy, severe/refractory; Pneumocystis pneumonia, adjunctive therapy for moderate to severe disease; Prostate cancer, metastatic, castration-resistant;

Prednicarbate: Dermatoses;

Prednisolone (Prednisolone sodium phosphate and prednisolone acetate forms): Corneal injury: Treatment of acute chemical injury of the cornea; Ophthalmic inflammnatory conditions: Treatment of ocular, anterior segment inflammation that is expected to be responsive to topical corticosteroid therapy; Allergic states: Control of severe or incapacitating allergic conditions intractable to adequate trials of conventional treatment in asthma, atopic dermatitis, drug hypersensitivity reactions, seasonal or perennial allergic rhinitis, and serum sickness; Dermatologic diseases: Bullous dermatitis herpetiformis; contact dermatitis; exfoliative erythroderma; exfoliative dermatitis; mycosis fungoides; pemphigus; severe erythema multiforme (Stevens-Johnson syndrome); severe psoriasis; severe seborrheic dermatitis; Endocrine disorders: Congenital adrenal hyperplasia; hypercalcemia associated with cancer; nonsuppurative thyroiditis; primary or secondary adrenocortical insufficiency; GI diseases: During acute episodes of Crohn disease or ulcerative colitis; Hematologic disorders: Acquired (autoimmune) hemolytic anemia; congenital (erythroid) hypoplastic anemia (Diamond-Blackfan anemia); erythroblastopenia (RBC anemia); immune thrombocytopenia (formerly known as idiopathic thrombocytopenic purpura), pure red cell aplasia; secondary thrombocytopenia; Neoplastic diseases: Treatment of acute leukemia and aggressive lymphomas; Nervous system: Acute exacerbations of multiple sclerosis; cerebral edema associated with primary or metastatic brain tumor, craniotomy, or head injury; Ophthalmic diseases: Allergic conjunctivitis; allergic corneal marginal ulcers; anterior segment inflammation; chorioretinitis; diffuse posterior uveitis and choroiditis; herpes zoster ophthalmicus; iritis and iridocyclitises; keratitis; optic neuritis; sympathetic ophthalmia; uveitis and other ocular inflammatory conditions unresponsive to topical corticosteroids; Renal disorders: To induce diuresis or remission of proteinuria in nephrotic syndrome, without uremia, of the idiopathic type or that due to lupus erythematosus; Respiratory diseases: Acute exacerbations of chronic obstructive pulmonary disease (COPD); allergic bronchopulmonary aspergillosis; aspiration pneumonitis; asthma; berylliosis; fulminating or disseminated pulmonary tuberculosis when used concurrently with appropriate antituberculous chemotherapy; hypersensitivity pneumonitis; idiopathic bronchiolitis obliterans with organizing pneumonia; idiopathic eosinophilic pneumonias; idiopathic pulmonary fibrosis; Loeffler syndrome (not manageable by other means); Pneumocystis carinii pneumonia (PCP) associated with hypoxemia occurring in an HIV-positive individual who is also under treatment with appropriate anti-PCP antibiotics; symptomatic sarcoidosis; Rheumatic disorders: As adjunctive therapy for short-term administration in acute and subacute bursitis, acute gout flares ; acute nonspecific tenosynovitis, ankylosing spondylitis, epicondylitis, polymyalgia rheumatica/temporal arteritis, posttraumatic osteoarthritis, psoriatic arthritis, relapsing polychondritis, rheumatoid arthritis (including juvenile rheumatoid arthritis), synovitis of osteoarthritis, acute rheumatic carditis, systemic lupus erythematosus, dermatomyositis/polymyositis, Sjogren syndrome, and certain cases of vasculitis; Miscellaneous: Acute or chronic solid organ rejection; trichinosis with neurologic or myocardial involvement; tuberculous meningitis with subarachnoid block or impending block, tuberculosis with enlarged mediastinal lymph nodes causing respiratory difficulty, tuberculosis with pleural or pericardial effusion (use appropriate antituberculous chemotherapy concurrently when treating any tuberculosis complications); Alcoholic hepatitis (severe); Asthma exacerbation; Bell palsy; Chronic obstructive pulmonary disease (COPD) (acute exacerbation);

Prednisone: Anti-inflammatory or immunosuppressant agent in the treatment of a variety of diseases, including allergic, hematologic (e.g., immune thrombocytopenia, warm autoimmune hemolytic anemia), dermatologic, GI, inflammatory, ophthalmic, neoplastic, rheumatic (e.g., acute gout flare, vasculitis, dermatomyositis, mixed cryoglobulinemia syndrome, polyarteritis nodosa, polymyositis, polymyalgia rheumatica, rheumatoid arthritis, systemic lupus erythematosus), autoimmune, nervous system (e.g., acute exacerbations of multiple sclerosis), renal, respiratory (e.g., asthma), and endocrine (e.g., primary or secondary adrenocorticoid deficiency); solid organ rejection (acute/chronic); Bell palsy, new onset; Chronic spontaneous urticaria, acute exacerbation; Duchenne muscular dystrophy; Giant cell arteritis, treatment; Graft-versus-host disease, acute, treatment; Hepatitis, autoimmune; Minimal change disease, treatment; Multiple myeloma (previously untreated; transplant-ineligible); Myasthenia gravis, crisis; Pericarditis, acute; Pneumocystis pneumonia, adjunctive therapy for moderate to severe disease; Prostate cancer, metastatic; Takayasu arteritis; Thyroiditis, subacute; Tuberculosis, pulmonary;

Triamcinolone (Triamcinolone acetonide form): Allergic rhinitis; Upper respiratory allergies; Acute bacterial rhinosinusitis, adjunct to antibiotics (empiric treatment); Chronic rhinosinusitis; Alopecia areata; discoid lupus erythematosus; keloids; localized hypertrophic, infiltrated, inflammatory lesions of granuloma annulate, lichen planus, lichen simplex chronicus (neurodermatitis), and psoriatic plaques; necrobiosis lipoidica diabeticorum; cystic tumors of an aponeurosis or tendon (ganglia); Allergic states: Control of severe or incapacitating allergic conditions intractable to adequate trials of conventional treatment in asthma, drug hypersensitivity reactions, perennial or seasonal allergic rhinitis, serum sickness, or transfusion reactions; Dermatologic diseases: Atopic dermatitis, bullous dermatitis herpetiformis, contact dermatitis, exfoliative erythroderma, mycosis fungoides, pemphigus, or severe erythema multiforme (Stevens-Johnson syndrome), vulvar dermatitis, psoriasis, seborrheic dermatitis; Endocrine disorders: Primary or secondary adrenocortical insufficiency, congenital adrenal hyperplasia, hypercalcemia associated with cancer, or nonsuppurative thyroiditis; GI diseases: To tide the patient over a critical period of disease in Crohn disease or ulcerative colitis; Hematologic disorders: Acquired (autoimmune) hemolytic anemia, Diamond-Blackfan anemia, pure red cell aplasia, select cases of secondary thrombocytopenia; Neoplastic diseases: Palliative management of leukemias and lymphomas; Nervous system: Acute exacerbations of multiple sclerosis; cerebral edema associated with primary or metastatic brain tumor or craniotomy; Ophthalmic diseases: Sympathetic ophthalmia, temporal arteritis, uveitis, and ocular inflammatory conditions unresponsive to topical corticosteroids; Renal diseases: To induce diuresis or remission of proteinuria in idiopathic nephrotic syndrome or that is caused by lupus erythematosus; Respiratory diseases: Berylliosis, fulminating or disseminated pulmonary tuberculosis when used concurrently with appropriate antituberculous chemotherapy, idiopathic eosinophilic pneumonias, symptomatic sarcoidosis; Rheumatic disorders: As adjunctive therapy for short-term administration in acute gout flares; acute rheumatic carditis; ankylosing spondylitis; psoriatic arthritis; RA, including juvenile RA; treatment of dermatomyositis, polymyositis, and systemic lupus erythematosus; Miscellaneous: Trichinosis with neurologic or myocardial involvement; tuberculous meningitis with subarachnoid block or impending block when used with appropriate antituberculous chemotherapy; Aphthous stomatitis;

Alclometasone (Alclometasone diproprionate form): Steroid-responsive dermatosis;

Betamethasone (Betamethasone, betamethasone sodium phosphate, betamethasone benzoate, betamethasone dipropionate, betamethasone valerate, and betamethasone acetate forms): Allergic states: Control of severe or incapacitating allergic conditions intractable to adequate trials of conventional treatment in asthma, atopic dermatitis, contact dermatitis, drug hypersensitivity reactions, perennial or seasonal allergic rhinitis, serum sickness, transfusion reactions; Dermatologic diseases: Bullous dermatitis herpetiformis, exfoliative erythroderma, mycosis fungoides, pemphigus, severe erythema multifortne (Stevens-Johnson syndrome); Endocrine disorders: Congenital adrenal hyperplasia, hypercalcemia associated with cancer, nonsuppurative thyroiditis. Synthetic analogs may be used in conjunction with mineralocorticoids where applicable; in infancy mineralocorticoid supplementation is of particular importance; Gastrointestinal diseases: During acute episodes in regional enteritis and ulcerative colitis; Hematologic disorders: Acquired (autoimmune) hemolytic anemia, Diamond-Blackfan anemia, pure red cell aplasia, selected cases of secondary thrombocytopenia; Neoplastic diseases: Palliative management of leukemias and lymphomas; Nervous system: Acute exacerbations of multiple sclerosis; cerebral edema associated with primary or metastatic brain tumor or craniotomy; Ophthalmic diseases: Sympathetic ophthalmia, temporal arteritis, uveitis and ocular inflammatory conditions unresponsive to topical corticosteroids; Renal diseases: To induce diuresis or remission of proteinuria in idiopathic nephrotic syndrome or that due to lupus erythematosus; Respiratoty diseases: Berylliosis, fulminating or disseminated pulmonary tuberculosis when used concurrently with appropriate antituberculous chemotherapy, idiopathic eosinophilic pneumonias, symptomatic sarcoidosis; Rheumatic disorders: Adjunctive therapy for short-term administration in acute gout flares; acute rheumatic carditis; ankylosing spondylitis; psoriatic arthritis; rheumatoid arthritis, including juvenile rheumatoid arthritis (selected cases may require low-dose maintenance therapy); treatment of dermatomyositis, polymyositis, and systemic lupus erythematosus; Miscellaneous: Trichinosis with neurologic or myocardial involvement, tuberculous meningitis with subarachnoid block or impending block when used with appropriate antituberculous chemotherapy; Adjunctive therapy for short-term administration in acute gout flares, acute and subacute bursitis, acute nonspecific tenosynovitis, epicondylitis, rheumatoid arthritis, synovitis of osteoarthritis; Treatment of alopecia areata; discoid lupus erythematosus; keloids; localized hypertrophic, infiltrated, inflammatory lesions of granuloma annulate, lichen planus, lichen simplex chronicus (neurodermatitis), and psoriatic plaques; necrobiosis lipoidica diabeticorum; Accelerate fetal lung maturation;

Betamethasone valerate and Betamethasone diproprionate administered together: Dermatoses: Relief of inflammatory and pruritic manifestations of corticosteroid-responsive dermatoses; Dermatoses of the scalp: Relief of inflammatory and pruritic manifestations of corticosteroid-responsive dermatoses of the scalp; Plaque psoriasis ('spray; patch): Treatment of mild to moderate plaque psoriasis in patients 18 years and older;

Clobetasol (Clobetasol propionate form): Steroid-responsive dermatoses;

Clobetasone (Clobetasone butyrate form): Dermatitis: Management of localized eczema and dermatitis including atopic eczema and irritant and allergic contact dermatitis;

Clocortolone (Clocortolone pivalate form): Steroid-responsive dermatoses;

Desoximetasone: Relief of inflammation and pruritic symptoms of corticosteroid-responsive dermatoses; Plaque psoriasis treatment;

Dexamethasone (Dexamethasone, dexamethasone phosphate, and dexamethasone sodium phosphate forms): Oral, IV; or IM injection: Anti-inflammatory or immunosuppressant agent in the treatment of a variety of diseases, including those of allergic, hematologic (e.g., immune thrombocytopenia), dermatologic, neoplastic, rheumatic, autoimmune, nervous system, renal, and respiratory origin; primary or secondary adrenocorticoid deficiency (not first line); management of shock, cerebral edema, and as a diagnostic agent; Intra-articular or soft tissue injection: As adjunctive therapy for short-term administration in synovitis of osteoarthritis, rheumatoid arthritis, acute and subacute bursitis, acute gouty arthritis, epicondylitis, acute nonspecific tenosynovitis, and posttraumatic osteoarthritis; Intralesional injection: Keloids; localized hypertrophic, infiltrated, inflammatory lesions of lichen planus, psoriatic plaques, granuloma annulate, and lichen simplex chronicus (neurodermatitis); discoid lupus erythematosus; necrobiosis lipoidica diabeticorum, alopecia areata; and cystic tumors of an aponeurosis or tendon (ganglia); Off-Label Use: Acute mountain sickness/high-altitude cerebral edema; Antiemetic regimens: chemotherapy-associated nausea and vomiting, prevention; Antiemetic regimens: radiation therapy-associated nausea and vomiting, prevention; Asthma, acute exacerbation; Coronavirus disease 2019 (COVID-19), treatment; Fetal lung maturation, acceleration of; Meningitis (bacterial), prevention of neurologic complications; Multiple myeloma;

Diflorasone (Diflorasone diacetate form): Dermatoses: Treatment of inflammation and pruritic symptoms of corticosteroid-responsive dermatoses;

Difluocortolone: Acute and chronic skin disease: Treatment of acute and chronic skin diseases responsive to the anti-inflammatory, antipruritic, and antiallergic effects of topical corticosteroids;

Fluticasone (Fluticasone propionate and fluticasone furoate forms): Asthma; Chronic obstructive pulmonary disease; Eosinophilic esophagitis (oral); Allergic rhinitis; Nasal polyps; Nonallergic rhinitis; Upper respiratory allergies; Acute bacterial rhinosinusitis, adjunct to antibiotics((empiric treatment); Chronic rhinosinusitis; Viral rhinosinusitis symptomatic relief; Dermatoses;

Halometasone: Treatment of steroid responsive skin disorders;

Mometasone (Mometasone furoate form): Corticosteroid-responsive dermatoses; Allergic rhinitis (seasonal and perennial) Nasal congestion associated with seasonal rhinitis; Nasal polyps; Seasonal allergic rhinitis (prophylaxis); Chronic rhinosinusitis; Rhinosinusitis, adjunctive treatment acute); Rhinosinusitis, treatment (acute, mild to moderate, uncomplicated); Asthma;

Rimexolone: Ophthalmic in, a iatoty conditions: Treatment of postoperative inflammation following ocular surgery; treatment of anterior uveitis;

Amcinonide: Relief of the inflammatory and pruritic manifestations of corticosteroid-responsive dermatoses;

Budesonide: Ulcerative colitis; Allergic rhinitis; Upper respiratoty symptoms: Relief of symptoms of hay fever or other upper respiratory allergies (e.g., nasal congestion, runny nose, itchy nose, sneezing); Nasal polyps; Rhinitis; Acute bacterial rhinosinusitis, adjunct to antibiotics (empiric treatment); Chronic rhinosinusitis; Asthma; Chronic obstructive pulmonary disease (acute exacerbation); Chronic obstructive pulmonary disease (stable); Eosinophilic esophagitis; Crohn disease, mild to moderate; Microscopic (lymphocytic and collagenous) colitis;

Ciclesonide: Seasonal and perennial allergic rhinitis; Acute bacterial rhinosinusitis, adjunct to antibiotics (empiric treatment); Chronic rhinosinusitis; Asthma;

Deflazacort: Duchenne muscular dystrophy;

Desonide: Atopic dermatitis; Corticosteroid-responsive dermatoses;

Flunisolide: Asthma; Rhinitis; Acute bacterial rhinosinusitis, adjunct to antibiotics (empiric treatment); Chronic rhinosinusitis; Non-allergic rhinitis; Symptomatic relief of viral rhinosinusitis;

Fluocinonide: Inflammatory and pruritic dermatologic conditions;

Halcinonide: Steroid-responsive dermatoses;

Cholesterol: Prevention/treatment of vitamin and mineral deficiencies;

Estradiol valerate: Breast cancer, metastatic; Hypoestrogenism (female); Osteoporosis prevention (female); Prostate cancer, advanced; Vasomotor symptoms associated with menopause; Vulvar and vaginal atrophy associated with menopause; Functional hypothalamic amenorrhea with low bone density (young adult females); Hormone therapy for transgender females (male-to-female);

Hydrocortisone (Hydrocortisone, hydrocortisone acetate ; hydrocortisone buteprate, hydrocortisone butyrate ; hydrocortisone succinate, and hydrocortisone valerate forms): Anal and genital pruritus, external; Corticosteroid-responsive dermatoses atopic dermatitis, contact dermatitis, vulvar dermatitis, psoriasis, seborrheic dermatitis); Hemorrhoids; Ulcerative colitis; Stasis dermatitis; Vaginitis, desquamative inflammatory; Allergic states: Control of severe or incapacitating allergic conditions intractable to adequate trials of conventional treatment in drug hypersensitivity reactions, perennial or seasonal allergic rhinitis, serum sickness, transfusion reactions, or acute noninfectious laryngeal edema; Dermatologic diseases: Atopic dermatitis; bullous dermatitis herpetiformis; contact dermatitis; exfoliative dermatitis; exfoliative erythroderma; pemphigus; severe erythema multiforme (Stevens-Johnson syndrome); severe psoriasis; severe seborrheic dermatitis; mycosis fungoides; Edematous states: To induce diuresis or remission of proteinuria in the nephrotic syndrome, without uremia, of the idiopathic type or that due to lupus erythematosus; Endocrine disorders: Acute adrenocortical insufficiency; congenital adrenal hyperplasia; hypercalcemia associated with cancer; nonsuppurative thyroiditis; primary or secondary adrenocorti cal insufficiency; preoperatively and in the event of serious trauma or illness, in patients with known adrenal insufficiency or when adrenocortical reserve is doubtful; shock unresponsive to conventional therapy if adrenocortical insufficiency exists or is suspected; GI diseases: To tide the patient over a critical period of the disease in ulcerative colitis and regional enteritis; Hematologic disorders: Acquired (autoimmune) hemolytic anemia; congenital (erythroid) hypoplastic anemia (Diamond Blackfan anemia); erythroblastopenia (RBC anemia); immune thrombocytopenia (formerly known as idiopathic thrombocytopenic purpura) in adults; pure red cell aplasia; select cases of secondary thrombocytopenia; Neoplastic diseases: Palliative management of leukemias and lymphomas (adults); acute leukemia of childhood; Nervous system: Cerebral edema associated with primary or metastatic brain tumor, or craniototny; Ophthalmic diseases: Severe acute and chronic allergic and inflammatory processes involving the eye, such as allergic conjunctivitis; allergic corneal marginal ulcers; anterior segment inflammation; chorioretinitis; diffuse posterior uveitis and choroiditis; herpes zoster ophthalmicus; iritis and iridocyclitis; keratitis; optic neuritis; sympathetic ophthalmia; other ocular inflammatory conditions unresponsive to topical corticosteroids; Respiratory diseases: Aspiration pneumonitis; bronchial asthma; berylliosis; fulminating or disseminated pulmonary tuberculosis when used concurrently with appropriate antituberculous chemotherapy; idiopathic eosinophilic pneumonias; Loeffler syndrome (not manageable by other means); symptomatic sarcoidosis; Rheumatic disorders: As adjunctive therapy for short-term administration in acute and subacute bursitis, acute gouty arthritis, acute nonspecific tenosynovitis, ankylosing spondylitis, epicondylitis, posttraumatic osteoarthritis, psoriatic arthritis, rheumatoid arthritis, including juvenile rheumatoid arthritis, synovitis of osteoarthritis; during an exacerbation or as maintenance therapy in acute rheumatic carditis, dermatomyositis (polymyositis), temporal arteritis, and systemic lupus erythematosus; Miscellaneous: Trichinosis with neurologic or myocardial involvement; tuberculous meningitis with subarachnoid block or impending block when used concurrently with appropriate antituberculous chemotherapy; In-hospital cardiac arrest; Septic shock; Thyroid storm;

Triamcinolone hexacetonide: Symptomatic treatment of subacute and chronic inflammatory joint diseases including: synovitis, tendinitis, bursitis, epicondylitis, rheumatoid arthritis (RA), juvenile idiopathic arthritis (JIA), osteoarthritis, or post-traumatic arthritis; and

Diflucortolone valerate: Acute and chronic skin disease.

Compounds having steroid core structures are also used in treating a wide variety of disorders in animals. For example, listed below are compounds having steroid core structures, and disorders they are used to treat in veterinary subjects, including dogs, cats, horses, swine, and cattle. In some embodiments, the composite particles described herein comprise the indicated compounds, and the disclosure provides a method of using such particles to treat the indicated disorders and/or for the following purposes in veterinary subjects:

Testosterone: Dogs—Testosterone-responsive urinary incontinence in neutered males; Dermatitis: bilateral alopecia. Cats—Testosterone-responsive urinary incontinence in neutered males;

Boldenone (Boldenone undecylenate form): Horses—as an aid for treating debilitated horses when an improvement in weight, haircoat, or general physical condition is desired;

Nandrolone: General Veterinary Patients—stimulate erythropoiesis in patients with certain anemias (e.g., secondary to renal failure, aplastic anemias);

Altrenogest: Horses—To suppress estrus or maintain pregnancy when progestin deficient. Swine—Synchronize estrus. Dogs—Luteal deficiency; prevent premature delivery;

Methyltestosterone: Female dogs—Treatment of estrogen-dependent tumors; Pseudopregnancy; Hormonal-dependent alopecias. Male dogs—Deficient libido; Testosterone-responsive incontinence; Certain hormonal alopecias. Cats—Hormonal-dependent alopecias; Increasing libido;

Stanozolol: Horses—Improve appetite, promote weight gain, and increase strength and vitality; treatment for chronic osteoarthritis. Dogs—Improve appetite, promote weight gain, and increase strength and vitality; Collapsing trachea. Cats—improve appetite, promote weight gain, and increase strength and vitality;

Mibolerone: Female dogs—Estrus prevention;

Danazol: Dogs—Treatment of canine immune-mediated thrombocytopenia and hemolytic anemia. Cats—Autoimmune hemolytic anemia and thrombocytopenia;

Osaterone acetate: Male dogs—Benign prostatic hypertrophy;

Spironolactone: Dogs—Potassium sparing diuretic or for adjunctive treatment for heart failure;

Estradiol: Horses—Enhancing estrus behavior and receptivity in ovariectomized mare. Dogs—

Estrogen-responsive urinary incontinence; Abortifacient, Cats—Abortifacient. Cattle—Abortifacient;

Megestrol (Megestrol acetate form): Female dogs—For postponement of estrus & the alleviation of false pregnancy; Male dogs—Benign prostatic hypertrophy. Cats—Many dermatologic & behavior-related conditions;

Estriol: Female dogs estrogen-responsive urinary incontinence in ovariohysterectomized female dogs;

A glepristone: Dogs—Pregnancy termination; Pyometra complex. Cats—Progesterone-dependent mammary hyperplasia;

Medroxyprogesterone (Medroxyprogesterone acetate form): Dogs Progestin-responsive dermatitis; aggressive behaviors; long-term reproductive control; treatment of young German shepherd dwarfs; short-term treatment of benign prostatic hypertrophy; luteal insufficiency. Cats—Sexually dimorphic behavior problems such as roaming, inter-male aggressive behaviors, spraying, and mounting; Feline psychogenic dermatitis and alopecia;

Trilostane: Dogs—Treatment of pituitary-dependent hyperadrenocorticism (PDH) and for the treatment of hyperadrenocorticism (HAC) associated with adrenocortical tumors (AT).

Cortisone (Cortisone acetate forms): Dogs—Oral treatment of hypoadrenocorticism;

Fluorometholone: General Veterinary Patients—Treatment of inflammation of the palpebral and bulbar conjunctiva, cornea, and anterior segment of the globe (blepharitis, conjunctivitis, keratitis, anterior uveitis);

Difluprednate: General Veterinary Patients—Treatment of inflammation following ocular injury or cataract surgery or to treat generalized inflammatory conditions of the anterior segment (conjunctivitis, keratitis, anterior uveitis);

Fludrocortisone (Fludrocortisone acetate form): Small Animals—Treatment of hypoadrenocorticism (Addison's disease); Adjunctive therapy in hyperkalemia;

Loteprednol: General Veterinary Patients—Treatment of inflammatory conditions of the palpebral and bulbar conjunctiva, cornea, and anterior segment of the globe (blepharitis, conjunctivitis, keratitis, anterior uveitis);

Methylprednisolone (methylprednisolone acetate and methylprednisolone succinate forms): General Veterinary Patients—Replacement of glucocorticoid activity in patients with adrenal insufficiency; Anti-inflammatory agent; Immunosuppressant;

PrednisoloneiPrednisone (Treated as bioequivalents): General Veterinary Patient—Replacement or supplementation (e,g., relative adrenal insufficiency associated with septic shock) for glucocorticoid deficiency secondary to hypoadrenocorticism; Anti-inflammatory agent; Immunosuppressant; Antineoplastic agent.

Triamcinolone (Triamcinolone acetonide form): General Veterinary Patients—Focal (e.g., pedal) or multifocal lesions fbr relatively short durations;

Betamethasone: General Veterinary Patients—Focal (e.g., pedal) or multifocal lesions for relatively short durations; Horses—intra-articular injection for treating pain and inflamed joints, Dogs—Induce premature labor;

Dexamethasone: General Veterinary Patients—Diagnostic agent to test for hyperadrenocorticism; Replacement or supplementation (e.g., relative adrenal insufficiency associated with septic shock) for glucocorticoid deficiency secondary to hypoadrenocorticism; Anti-inflammatory agent; Immunosuppression; Antineoplastic agent;

Flumethasone: Horses—Musculoskeletal conditions due to inflammation, where permanent structural changes do not exist, such as bursitis, carpitis, osselets, and myositis; Allergic states such as urticaria (hives) and insect bites. Dogs—Musculoskeletal conditions due to inflammation of muscles or joints and accessory structures, where permanent structural changes do not exist, such as arthritis, osteoarthritis, intervertebral disc syndrome and myositis; Certain acute and chronic dermatoses of varying etiology to help control the pruritus, irritation, and inflammation associated with these conditions; Allergic states such as urticaria and insect bites. Cats—Certain acute and chronic dermatoses of varying etiology to help control the pruritus, irritation, and inflammation associated with these conditions;

Fluticasone (Fluticasone propionate form): Horses—Recurrent airway obstruction or inflammatory airway disease. Dogs—Chronic cough. Cats—Feline asthma;

Mometasone (Mometasone furoate form): General Veterinary Patient—Focal (e.g., pedal) or multifocal lesions and for relatively short durations;

Rimexolone: General Veterinary Patients—Symptomatic relief of corticosteroid-responsive inflammatory conditions of the palpebral and bulbar conjunctiva, cornea, and anterior segment of the globe (e.g., allergic conjunctivitis, acne rosacea, superficial punctate keratitis, iritis, and cyclitis). Horses—Treatment of uveitis;

Budesonide: Small Animals—Treatment of inflammatory intestinal diseases; dermatitis; Corticosteroid-responsive dermatoses;

Deoxycorticosterone: Dogs—Parenteral treatment of adrenocortical insufficiency (Addison's disease). Cats—Parenteral treatment of adrenocortical insufficiency (Addison's disease);

Alfaxalone: Dogs—Induction and maintenance of anesthesia and for induction of anesthesia followed by maintenance with an inhalant anesthetic. Cats—Induction and maintenance of anesthesia and for induction of anesthesia followed by maintenance with an inhalant anesthetic;

Hydrocortisone: General Veterinary Patient—Focal (e.g., pedal) or multifocal lesions for relatively short durations; When an acute glucocorticoid/mineralocorticoid effect is desired (e.g., acute adrenal insufficiency; critical illness-related corticosteroid insufficiency [CIRCI]); Inflammatory conjunctivitis;

Desoxycorticosterone (Desoxycorticosterone pivalate form): Dogs—Treatment of hypoadrenocorticism (Addison's disease). Cats—Treatment of hypoadrenocorticism (Addison's disease); and

Isoflupredone acetate: Horses—Anti-inflammatory & immunosuppressive effects; Swine—Anti-inflammatory & immunosuppressive effects; Cattle—Anti-inflammatory & immunosuppressive effects.

Accordingly, in one aspect, the disclosure provides a method of treating a liver disease or a peroxisomal disorder in a subject in need of treatment, comprising administering to the subject a therapeutically effective amount of a plurality of composite particles described herein, such as a pharmaceutical composition comprising a plurality of composite particles described herein (e.g., composite particles comprising a bile acid or a salt or ester thereof).

For example, in one aspect, the disclosure provides a method of treating a liver disease in a subject in need of treatment, comprising administering to the subject a therapeutically effective amount of a plurality of composite particles described herein, such as a pharmaceutical composition comprising a plurality of composite particles described herein (e.g., composite particles comprising a bile acid or a salt or ester thereof). In some embodiments, the liver disease is a bile acid synthesis disorder. In some embodiments, the liver disease is a bile acid synthesis disorder due to a single enzyme defect. For example, in some embodiments, the single enzyme defect in the bile acid synthesis disorder is a 3β-hydroxy-Δ5-C27-steroid oxidoreductase deficiency, alpha-methylacyl-CoA racemase (AMACR) deficiency, amino acid N-acyltransferase deficiency, bile acid CoA ligase deficiency, cholesterol 7α-hydroxylase deficiency, Δ4-3-oxosteroid 5β-reductase deficiency, oxysterol 7α-hydroxylase deficiency, sterol 27-hydroxylase deficiency (cerebrotendinous xanthomatosis), or trihydroxycholestanoic acid CoA oxidase deficiency. In other embodiments, the liver disease is selected from primary biliary cholangitis, primary sclerosing cholangitis, bile duct stones, and non-alcoholic fatty liver disease. In some embodiments, the liver disease is primary biliary cholangitis:

In some embodiments, the disclosure provides a method of treating a peroxisomal disorder in a subject in need of treatment, comprising administering to the subject a therapeutically effective amount of a plurality of composite particles described herein, such as a pharmaceutical composition comprising a plurality of composite particles described herein. In some embodiments, the peroxisomal disorder is a Zellweger spectrum disorder. Zellweger spectrum disorders are a group of autosomal recessive genetic disorders caused by mutations in PEX genes that encode peroxins; subdivisions of the spectrum are Zellweger syndrome, neonatal adrenoleukodystrophy, and infantile Refsum disease. In some embodiments, the subject is suffering from a Zellweger spectrum disorder with manifestations of liver disease, steatorrhea, or complications from decreased fat soluble vitamin absorption.

In some embodiments, the disclosure provides a method of treating a disorder selected from cardiometabolic disease, gallstones, type-2 diabetes, human immunodeficiency virus type 1 (HIV-1), and acute pancreatitis, comprising administering to the subject a therapeutically effective amount of a plurality of composite particles described herein, such as a pharmaceutical composition comprising a plurality of composite particles described herein (e.g., composite particles comprising a bile acid or a salt or ester thereof).

In one aspect, the disclosure provides a method of non-surgical removal of a localized fat deposit in a subject, comprising contacting the deposit with an effective amount of a plurality of composite particles described herein, such as a pharmaceutical composition comprising a plurality of composite particles described herein (e.g., composite particles comprising a bile acid or a salt or ester thereof). In some embodiments, the subject has a localized fat deposit and desires to remove the deposit. In some embodiments, the localized fat deposit is located in the submental region of the subject. In some embodiments, the localized fat deposit is located in the abdominal region of the subject. In some embodiments, the deposit is contacted with the composition by subcutaneous injection.

In an aspect, the disclosure provides a method of reducing a subcutaneous fat deposit in a subject in need thereof, comprising administering locally to the subcutaneous fat deposit in the subject an effective amount of a plurality of composite particles described herein, such as a pharmaceutical composition comprising a plurality of composite particles described herein (e.g., composite particles comprising a bile acid or a salt or ester thereof). In some embodiments, the subject has a subcutaneous fat deposit and desires to remove the deposit. In some embodiments, the subcutaneous fat deposit is located in the submental region of the subject. In some embodiments, the subcutaneous fat deposit is located in the abdominal region of the subject. In some embodiments, the deposit is contacted with the composition by subcutaneous injection. In some embodiments, the subcutaneous fat deposit is associated with a condition selected from the group consisting of obesity, fat redistribution syndrome, eyelid fat herniation, lipomas, Dercum's disease, lipodystrophy, buffalo hump lipodystrophy, dorsocervical fat, visceral adiposity, breast enlargement, hyperadiposity, diffused body fat around trunk and arms, and fat deposits associated with cellulite.

In some embodiments, the disclosure provides a method of treating cancer in a subject in need thereof, comprising administering to the subject a therapeutically effective amount of a plurality of composite particles described herein, such as a pharmaceutical composition comprising a plurality of composite particles described herein (e.g., composite particles comprising a bile acid or a salt or ester thereof). In some embodiments, the cancer is selected from colorectal cancer, cervical cancer, gastric cancer, and liver cancer. In some embodiments, the cancer is colorectal cancer.

In some embodiments, the disclosure provides a method of reducing the proliferation of cancer cells, comprising contacting the cells with an effective amount of a plurality of composite particles described herein, such as a pharmaceutical composition comprising a plurality of composite particles described herein (e.g., composite particles comprising a bile acid or a salt or ester thereof). In some embodiments, the cancer cells are selected from colorectal cancer, cervical cancer, gastric cancer, and liver cancer cells. In some embodiments, the cancer cells are colorectal cancer cells.

In some embodiments, the disclosure provides a method of treating a disorder in a subject, wherein the disorder is selected from the group consisting of endocrine disorders, rheumatic disorders, collagen diseases, dermatologic diseases, allergic states, ophthalmic diseases, respiratory diseases, hematologic disorders, neoplastic diseases, gastrointestinal diseases, nervous system disorders, inflammatory disorders, renal diseases, comprising administering to the subject a therapeutically effective amount of a plurality of composite particles described herein, such as a pharmaceutical composition comprising a plurality of composite particles described herein (e.g., composite particles comprising a corticosteroid or a salt or ester thereof).

In some embodiments, the disclosure provides a method of treating a respiratory disease in a subject in need thereof, comprising administering to the subject a therapeutically effective amount of a plurality of composite particles described herein, such as a pharmaceutical composition comprising a plurality of composite particles described herein (e.g., composite particles comprising a corticosteroid or a salt or ester thereof). In some embodiments, the respiratory disease is selected from asthma, croup, chronic obstructive pulmonary disease (COPD), bronchitis, and pneumonia (e.g., interstitial pneumonia).

The disclosed methods involve administration of an “effective amount” or a “therapeutically effective amount” of the composite particles. As used herein, both terms refer to an amount effective, at dosages and for periods of time necessary, to achieve the desired result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound (e.g., a component of the composite particles or the compositions) are outweighed by the therapeutically beneficial effects. For example, a therapeutically effective amount of the composite particles described herein may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/g, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 m/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The disclosed methods may further comprise a step of administering one or more additional therapeutic agents to the subject. The composite particles and the additional therapeutic agent(s) may be administered to the subject simultaneously or sequentially. In some embodiments, the additional therapeutic agent or agents may be administered in the same composition as the composite particles. In other embodiments, there may be an interval of time between administration of the additional therapeutic agent(s) and the composite particles. In some embodiments, administration of an additional therapeutic agent with the composite particles may allow lower doses of the other therapeutic agents and/or administration at less frequent intervals. When used in combination with one or more other active ingredients, the composite particles and the other active ingredients may be used in lower doses than when each is used singly.

For example, the methods may further comprise a step of administering an additional therapeutic agent to the subject, wherein the additional therapeutic agent is selected from anti-inflammatory agents, analgesics, chemotherapy agents, and bile acids or salts thereof.

Anti-inflammatory agents suitable for use with the disclosed compositions and methods can include both steroidal anti-inflammatory agents and non-steroidal anti-inflammatory agents. Suitable steroidal anti-inflammatory agents include, but are not limited to, corticosteroids such as hydrocortisone, dexamethasone, dexamethasone phosphate, beclomethasone dipropionate, clobetasol valerate, desonide, desoxymethasone, desoxycorticosterone acetate, dichlorisone, diflorasone diacetate, diflucortolone valerate, fluadrenolone, fluclarolone acetonide, fludrocortisone, fludrocortisone acetate, flumethasone pivalate, fluosinolone acetonide, fluocinonide, flucortine butylester, fluocortolone, fluprednidene (fluprednylidene)acetate, flurandrenolone, halcinonide, hydrocortisone, hydrocortisone acetate, hydrocortisone butyrate, methylprednisolone, triamcinolone, triamcinolone acetonide, cortisone, cortodoxone, flucetonide, difluorosone diacetate, fluradrenalone acetonide, medrysone, amciafel, am.cinafide, betamethasone and the balance of its esters, chlorprednisone, chlorprednison.e acetate, clocortelone, clescinolone, dichlorisone, difluprednate, flucioronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, and mixtures thereof. Pharmaceutically acceptable salts and esters of these agents may also be used.

Suitable non-steroidal anti-inflammatory agents include, but are not limited to: oxicams, such as piroxicam, isoxicam, tonexicam, sudoxicam, and CP-14,304; salicylates, such as salicylic acid, aspirin, disalcid, benotylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; fenamates, such as mefenamic, meclofenamic, flufenamic, niflumic, and tolfenamic acids; propionic acid derivates, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and pyrazoles, such as phenybutazone, oxyphenbutazone, feprazone, azapropazone, and trimethazone; and mixtures of any thereof. Pharmaceutically acceptable salts and esters of these agents may also be used.

Analgesics may reduce discomfort due to inflammation, particularly after parenteral administration (e.g., subcutaneous injection) of a composition of the disclosure. Suitable analgesics include, but are not limited to, injectable local amine and ester anesthetics, such as lidocaine, mepivacaine, bupivacaine, procaine, chloroprocaine; etidocaine, prilocaine and tetracaine. Mixtures of these analgesics, as well as the pharmaceutically acceptable salts and esters or these agents, may also be used.

Bile acids or salts thereof may also be used in combination with the composite particles. The separately administered bile acid or salt or ester thereof may be the same as or different from the bile acid or salt or ester thereof that is present in the composite particles. Exemplary bile acids include cholic acid, deoxycholic acid, chenodeoxycholic acid, lithocholic acid, glycocholic acid, taurocholic acid, glycodeoxycholic acid, taurodeoxycholic acid, glycochenodeoxycholic acid, taurochenodeoxycholic acid, glycolithocholic acid, taurolithocholic acid, ursodeoxycholic acid, glycoursodeoxycholic acid, and tauroursodeoxycholic acid. For example, obeticholic acid is often used in combination with ursodeoxycholic acid for treatment of primary biliary cholangitis. Accordingly, in some embodiments, when the composite particles comprise obeticholic acid, the methods further comprise administration of ursodeoxycholic acid. In other embodiments, when the composite particles comprise ursodeoxycholic acid, the methods further comprise administration of obeticholic acid.

In some embodiments, the composite particles may be used in combination with a chemotherapy agent. Exemplary chemotherapy agents include those listed in the “A to Z List of Cancer Drugs” published by the National Cancer Institute.

The following examples further illustrate aspects of the disclosure but, of course, should not be construed as in any way limiting its scope.

### EXAMPLES

### Materials and Methods

All materials were purchased from Sigma-Aldrich and used as received unless indicated otherwise.

Microparticle Characterization: Microparticles were imaged with brightfield and scanning electron microscopy (SEM) (JEOL JSM-7800FLV) microscopes. Fluorescent images of the particles were acquired using an inverted Nikon Microscope and via fluorescent lamps and a TRITC/CY3 filter. For the SEM imaging, the samples were dried on a glass slide and coated with carbon and imaged with both secondary scattered and backscattered probes. Energy-dispersive spectroscopy (EDS) analyses were used to characterize the elemental composition of the particles. EDS analysis was performed on the microparticles by an Oxford XMaxN 80 mm² silicon-drift energy-dispersive X-ray spectrometer. Dynamic light scattering (DLS) was used to determine the hydrodynamic diameter of particles and zeta potential (ZP) was used to determine particle surface charge. DLS and ZP analyses were performed on the particles with a Malvern Nano-ZS zetasizer.

### Example 1

### Preparation of Composite Microparticles with Gold Ions

Method 1: 10 mg gold(III) chloride was dissolved in 1 mL ethyl acetate. This ethyl acetate solution was emulsified with 2 mL water via vortexing for 15 seconds. The resulting oil/water emulsion was added to 10 mL of a 0.3 wt % sodium cholate solution in a 100 mL beaker, and the mixture was stirred at 300 rpm. Composite particles appeared after 1-3 minutes of stirring. Particles were isolated via centrifugation at 2000 rpm for 5 minutes.

Method 2: Excess sodium cholate (˜5 mg) was added to 1 mL ethyl acetate. Due to the low solubility of sodium cholate in ethyl acetate, the solution was centrifuged down to form a pellet of excess sodium cholate, and the supernatant was collected. 10 mg gold(III) chloride was added to the collected ethyl acetate/sodium cholate supernatant solution, and the resulting solution was emulsified with 2 mL water via vortexing for 15 seconds. The resulting oil/water emulsion was added to 10 mL of a 0.3 wt % sodium cholate solution in a 100 mL beaker, and the mixture was stirred at 300 rpm. Composite particles appeared immediately. Particles were isolated via centrifugation at 2000 rpm for 5 minutes.

Method 3: 10 mg gold(III) chloride was dissolved in 4 mL water. This solution was emulsified with 1. mL ethyl acetate via vortexing for 15 seconds. The resulting oil water emulsion was added to 10 mL of a 0.3 wt % sodium cholate solution in a 100 mL beaker, and the mixture was stirred at 300 rpm. Composite particles appeared after 1-3 minutes of stirring. Particles were isolated via centrifugation at 2000 rpm for 5 minutes.

In addition to the sodium cholate particles prepared according to Methods 1-3 above, particles were also prepared according to Method 1, replacing the sodium cholate with sodium deoxycholate, methylprednisolone sodium succinate, and hydrocortisone sodium succinate.

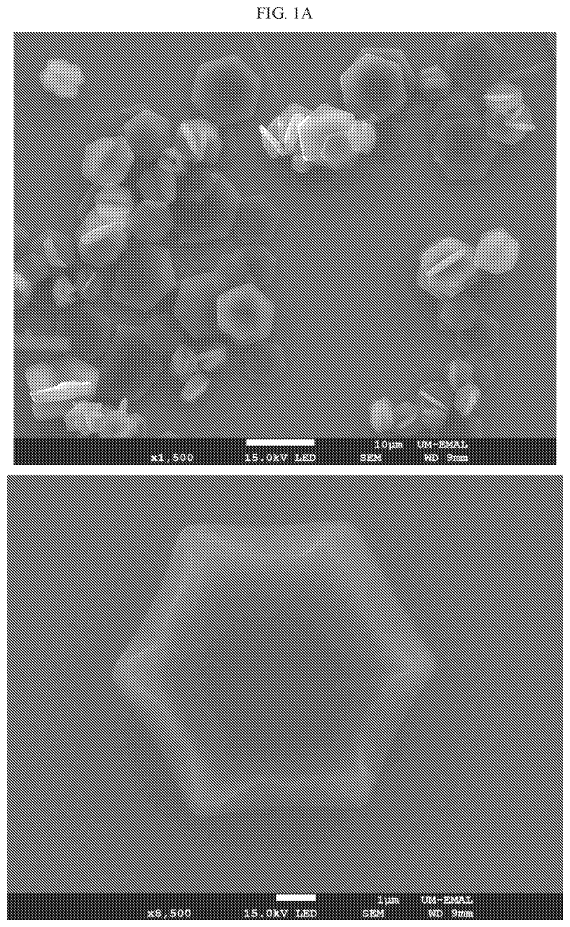

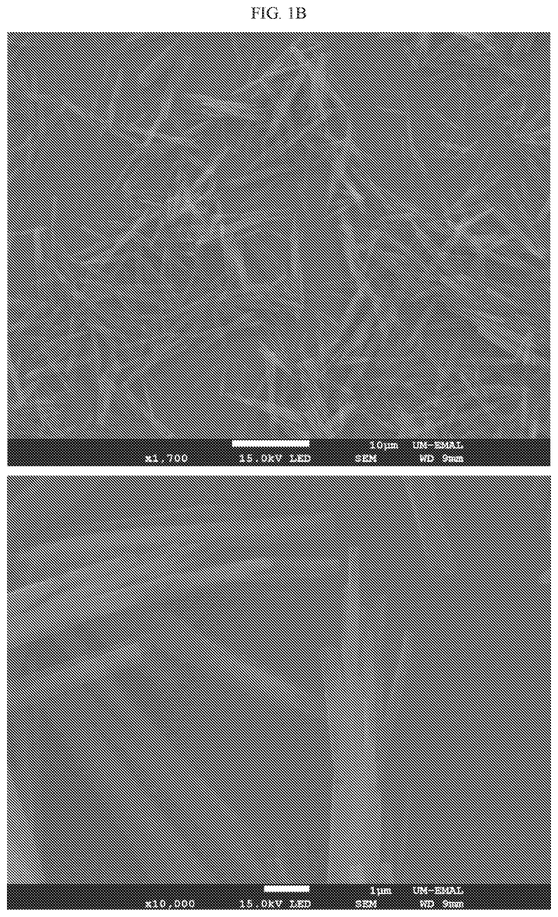

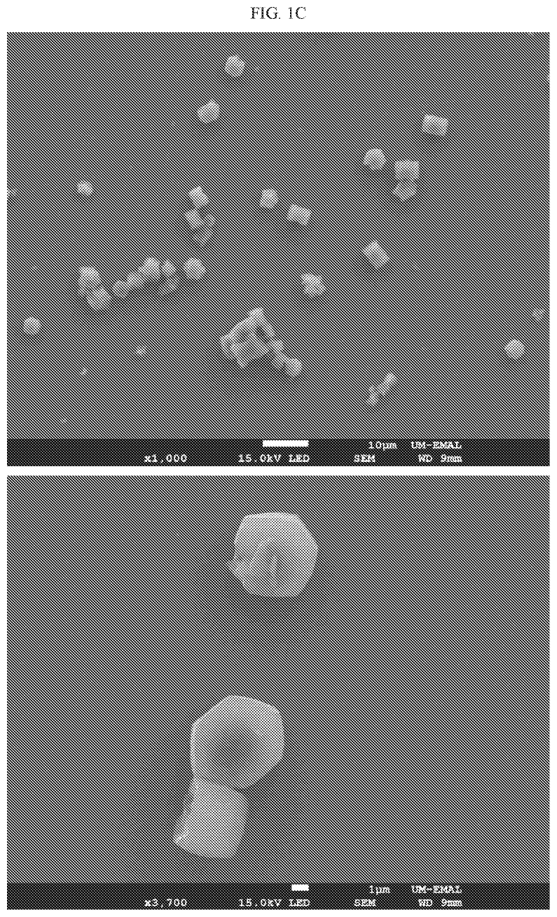

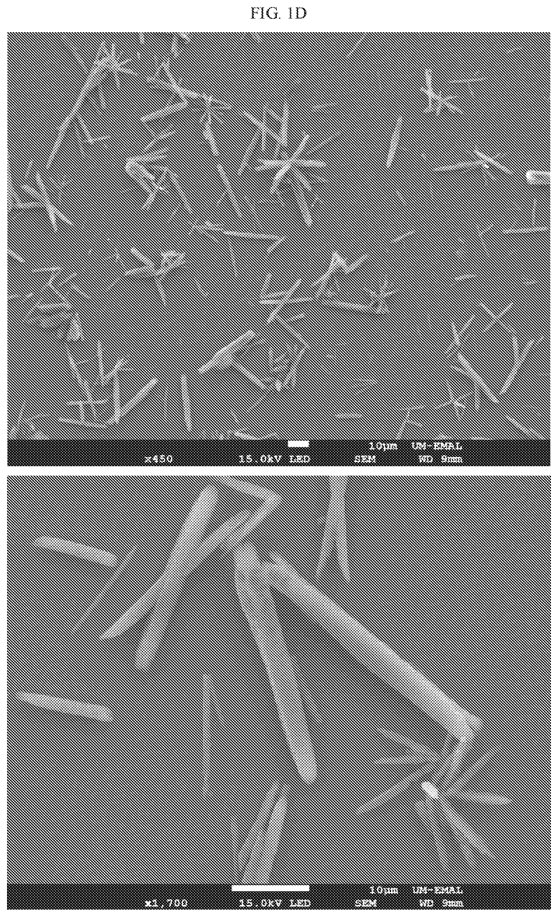

SEM images of the particles are shown in FIGS. 1A-1D. Specifically: FIG. 1A shows SEM images of the cholate particles prepared according to Method 1, at 1500× magnification (top) and 8500× magnification (bottom); FIG. 1B shows SEM images of the deoxycholate particles prepared according to Method 1, at 1700× magnification (top) and 10000× magnification (bottom); FIG. 1C shows SEM images of the methylprednisolone particles prepared according to Method 1, at 1000× magnification (top) and 3700× magnification (bottom); and FIG. 1D shows SEM images of the hydrocortisone particles prepared according to Method 1, at 450× magnification (top) and 1700× magnification (bottom).

EDS data for the particle shown in the lower panel of FIG. 1A is shown in FIG. 2. This particle is primarily composed of carbon and oxygen, with trace amounts of gold, sodium, and chlorine. The large silicon peak is due to the signal obtained from the silicon wafer on which the sample is mounted. The data confirm that gold is contained within the particles.

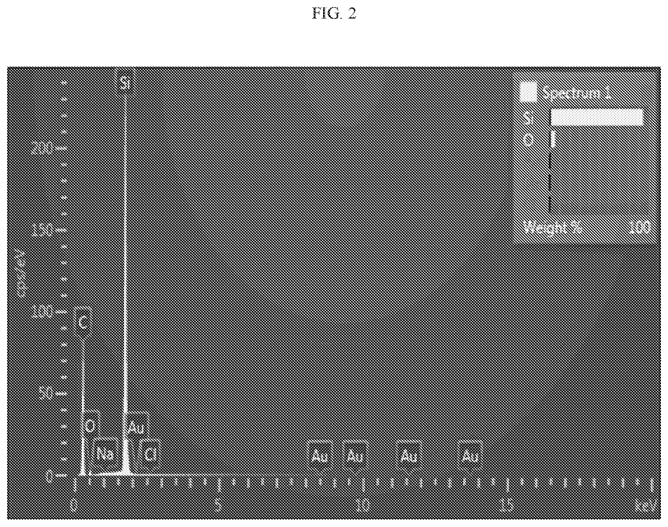

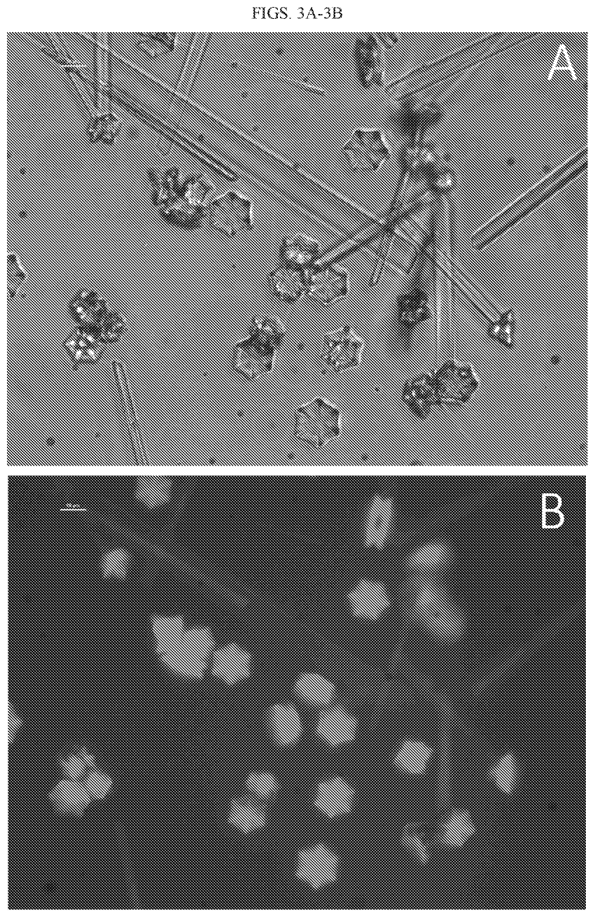

Rhodamine B-loaded cholate particles were prepared according to Method 1, with 3 mg rhodamine B introduced in the oil phase of the oil/water emulsion (i.e. the solution of gold(III) chloride and ethyl acetate). Images of the resulting particles are shown in FIG. 3A (brightfield microscopy image at 40× magnification); and FIG. 3B (the same particles fluorescing when exposed to light at 546 nm, confirming that rhodamine B was loaded in to the hexagonal cholate/gold ion particles). Rhodamine B loaded particles were also prepared by adding the rhodamine B to the water phase of the oil/water emulsion.

### Example 2

### Preparation of Composite Microparticles with Iron Ions

Method 5-10 mg iron(II) sulfate heptahydrate was dissolved in 50 μL water. This solution was added to 10 mL of a 0.5 wt % solution of dexamethasone phosphate sodium salt in water with stirring at 500 rpm. Stirring was continued for 15 minutes, with particles beginning to form soon after stirring began. Particles were isolated via centrifugation at 15000 rpm for 5 minutes. The pellet was resuspended in water and the centrifugation was repeated 3 more times to wash the particles.

Method 2: 10 mg iron(II) sulfate heptahydrate was dissolved in 50 μL water. This solution was vortexed into 1 mL ethyl acetate for 15 seconds. To this mixture was added 2 mL of a 1 wt % solution of dexamethasone phosphate sodium salt in water, and this mixture was vortexed for 15 seconds. To this mixture was added 10 mL of a 0.5 wt % solution of dexamethasone phosphate sodium salt in water followed by mixing. Stirring at 500 rpm was conducted for 5-15 minutes, with particles beginning to form soon after stirring began. Particles were isolated via centrifugation at 15000 rpm for 5 minutes. The pellet was resuspended in water and the centrifugation was repeated 3 more times to wash the particles.

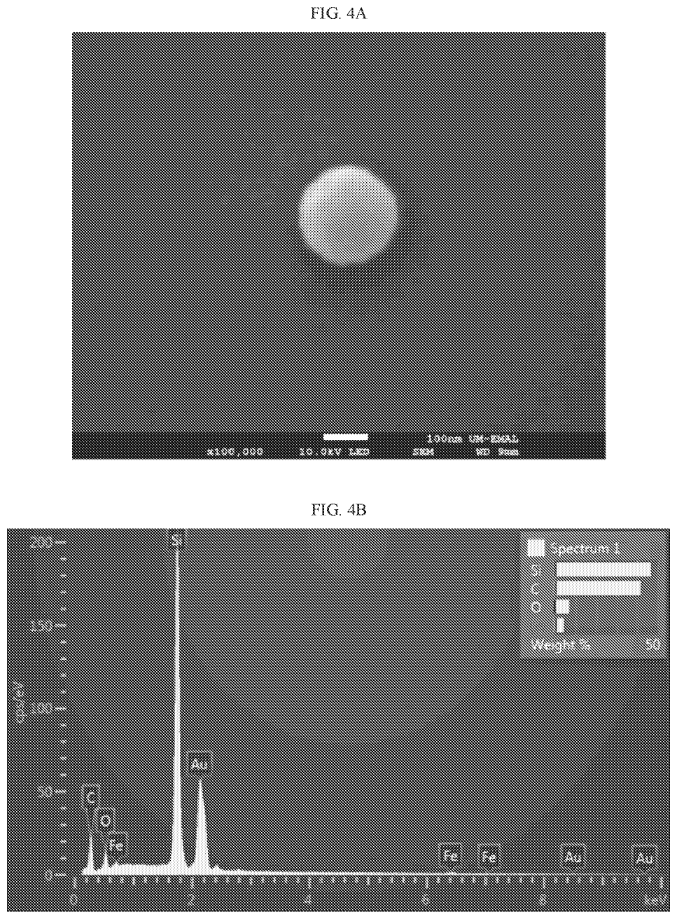

An SEM image of a representative particle is shown in FIG. 4A. Specifically: FIG. 4A shows an SEM image of the dexamethasone particles prepared according to Method 1. at 100,000× magnification. EDS data for the particle shown in FIG. 4A is shown in FIG. 4B. This particle is composed of carbon and oxygen, with trace amounts of iron. The large silicon peak is due to the signal obtained from the silicon wafer on which the sample is mounted. The large gold peak is due to the signal obtained from gold coating particles for SEM imaging. The data confirm that iron is contained within the particles.

Dynamic light scattering (DLS) data for particles prepared according to Method 1 using 5 mg or 10 mg iron sulfate heptahydrate showed that the mean particle diameter was 165 nm and 205 nm respectively. Zeta potential was determined for particles prepared according to Method 1 using 5 mg or 10 mg iron sulfate heptahydrate, and was found to be −25.9 mV and −16.2 my respectively. These data suggest that particle surface charge can be modulated by adjusting the iron ion concentration.

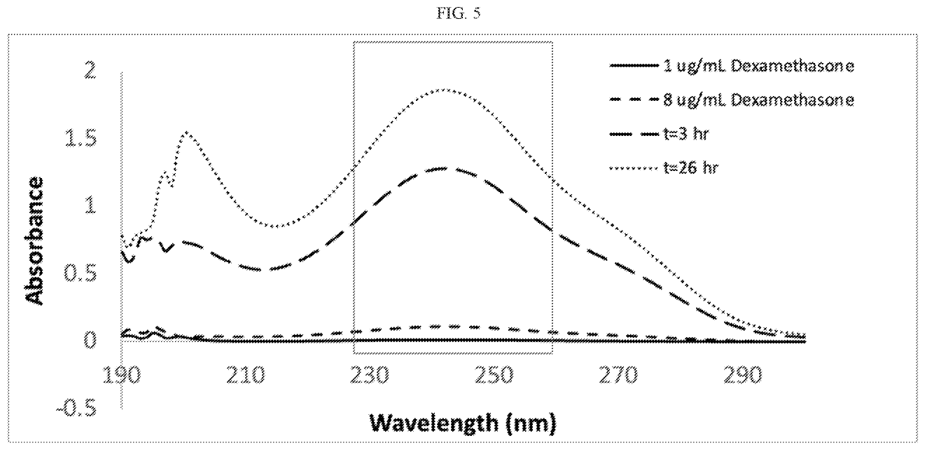

Dexamethasone release from particles prepared according to Method 1 was evaluated by suspending 6.9 mg particles in 42 mL phosphate-buffered saline (PBS) and rotating the mixture at 37 C for 26 hours. Samples of the liquid were collected and analyzed by UV-visible spectrometry compared to dexamethasone standards (either 1 μg/mL dexamethasone phosphate or 8 μg/mL dexamethasone phosphate. The data are shown in FIG. 5. The absorbance between about 230-260 nm shows the release of dexamethasone into the solution.

Rhodatnine B-loaded dexamethasone particles were prepared according to Method 1, with 2 mg rhodamine B introduced in the 50 μL water/iron(II) sulfate heptahydrate solution. The particles were pink in color and fluoresced, similar to the cholate/gold ion particles described in Example 1, suggesting that the particles were loaded with rhodamine.

### Example 3

### Preparation of Composite Microparticles with Acids

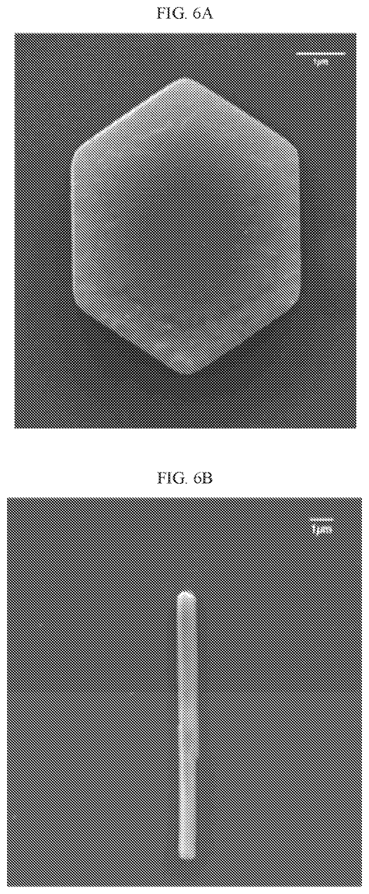

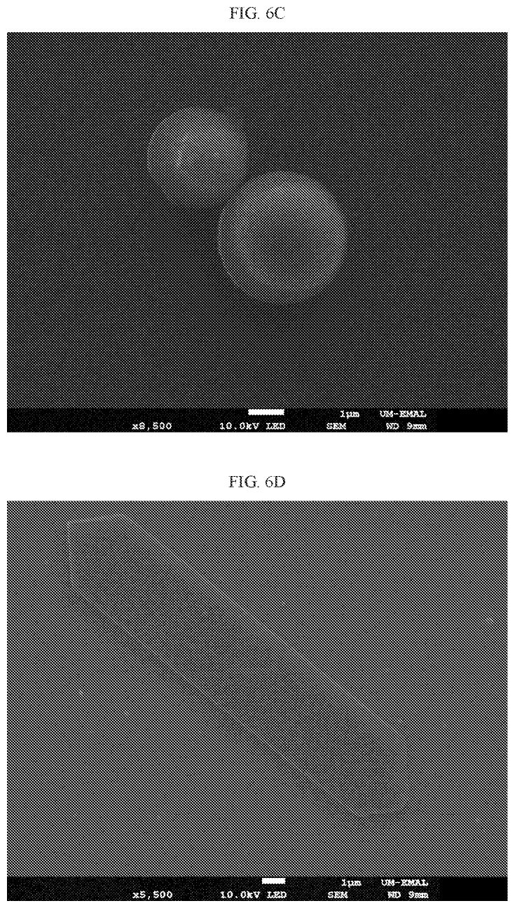

Bile Salt/HCl Particles. Hydrochloric acid was added to water until a pH of 2, and then 50 mg of a bile acid salt (sodium cholate, sodium deoxycholate, sodium ursodeoxycholate, or sodium chenodeoxycholate) was added to 2 mL of pH 2 water and the solution was vortexed until clear. 2 mL of this solution was added to 1 mL of ethyl acetate, and the mixture was vortexed for 15 seconds. To this emulsion was added 10 mL water at pH 3 (adjusted with HQ). Further stirring at 300 rpm for 1-2 minutes was required for particle formation. SEM images of particles formed using sodium cholate, sodium deoxycholate, sodium chenodeoxycholate, and sodium ursodeoxycholate are shown in FIGS. 6A, 6B, 6C, and 6D, respectively. EDS data for the sodium chenodeoxycholate and sodium ursodeoxycholate particles showed peaks only for carbon, oxygen, and silicon (with the Si peak resulting from the Si wafer on which the sample is mounted).

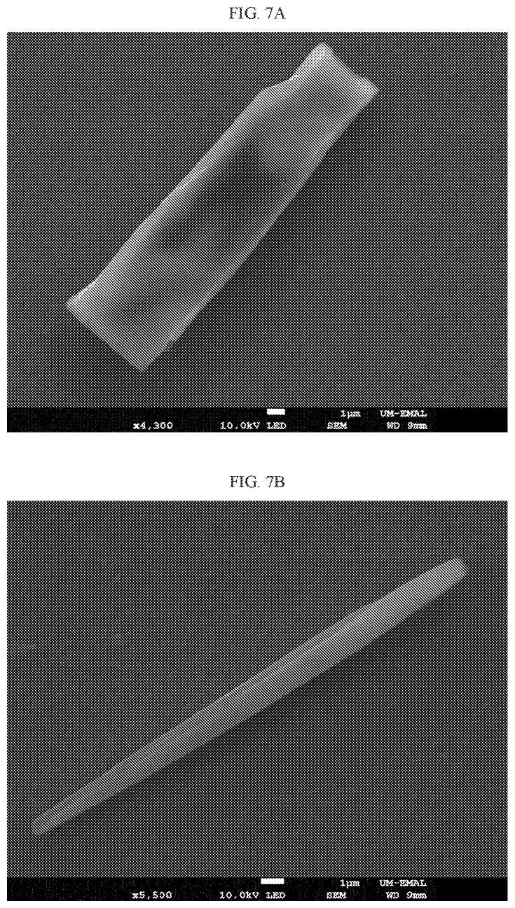

Corticosteroid/HCl Particles. Hydrochloric acid was added to water until a pH of 3, and then 50 mg of a corticosteroid compound (methylprednisolone succinate or hydrocortisone succinate) was added to 2 mL of pH 3 water and the solution was vertexed until clear. 2 mL of this solution was added to 1 mL of ethyl acetate, and the mixture was vortexed for 15 seconds. To this emulsion was added 10 mL water at pH 2 (adjusted with HCl). Further stirring at 300 rpm for 1-2 minutes was required for particle formation. SEM images of particles formed using methylprednisolone succinate and hydrocortisone succinate are shown in FIGS. 7A and 7B, respectively. EDS data for these particles showed peaks only for carbon, oxygen, and silicon (with the Si peak resulting from the Si wafer on which the sample is mounted); notably, chlorine was not detected.

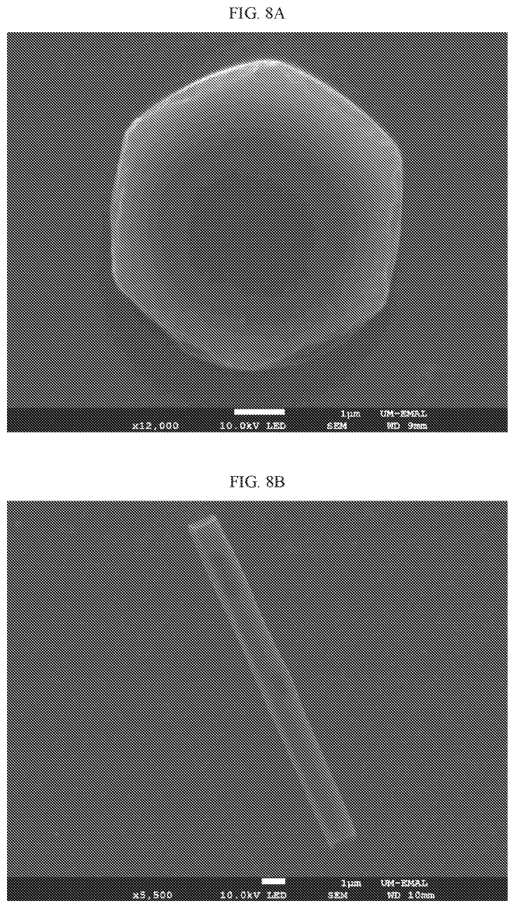

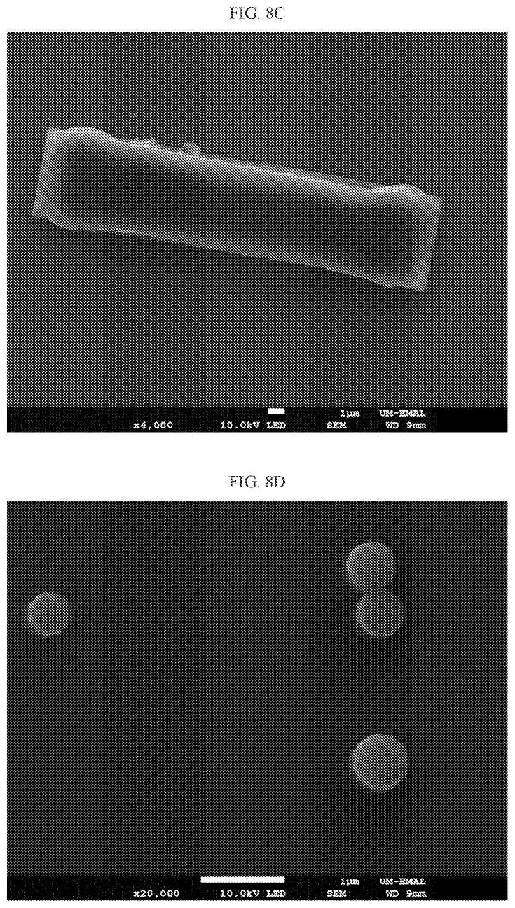

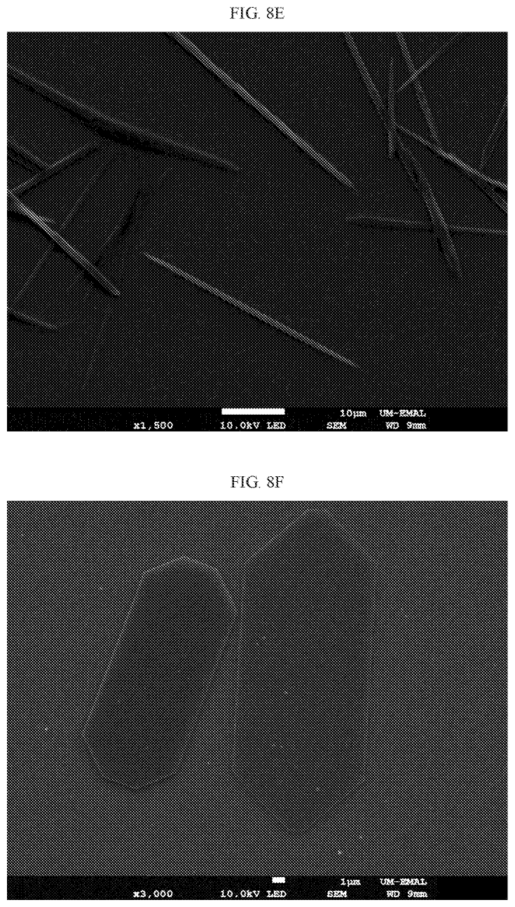

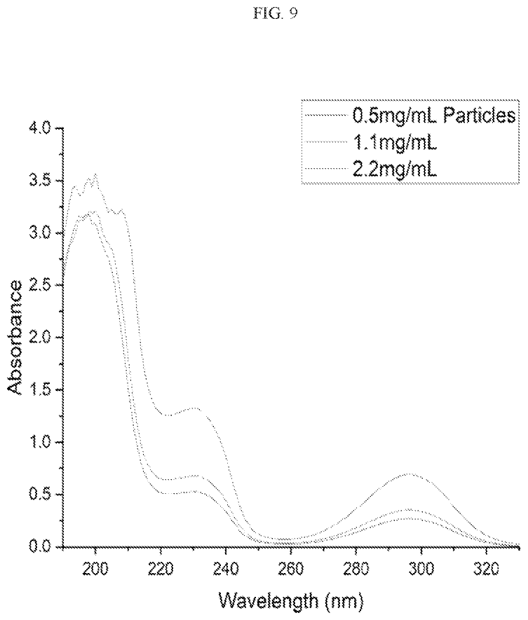

Bile Salt or Corticosteroid/Salicylic Acid Particles. 5-6 mg salicylic acid was dissolved in 1 mL ethyl acetate. To this solution was added a 1 wt % bile acid salt or corticosteroid (sodium cholate, sodium deoxycholate, methylprednisolone succinate, hydrocortisone succinate, sodium ursodeoxycholate, or sodium chenodeoxycholate) was added and the mixture was vortexed for 15 seconds. This emulsion was added to 10 mL of a 0.3 wt % solution of the same bile acid salt or corticosteroid (sodium cholate, sodium deoxycholate, methylprednisolone succinate, hydrocortisone succinate, sodium ursodeoxycholate, or sodium chenodeoxycholate). Further stirring at 300 rpm for 1 minute was required for particle formation. SEM images of particles formed using sodium cholate, sodium deoxycholate, methylprednisolone succinate, sodium chenodeoxycholate, hydrocortisone succinate, and sodium ursodeoxycholate are shown in FIGS. 8A, 8B, 8C, 8D, 8E, and 8F, respectively. EDS data for the methylprednisolone succinate, sodium chenodeoxycholate, hydrocortisone succinate, and sodium ursodeoxycholate particles showed peaks only for carbon, oxygen, and silicon (from the Si wafer on which the sample is mounted).

Salicylic acid release from the deoxycholate particles was determined by spectrometry, measuring the absorbance at 296 nm. The deoxycholate particles were fully degraded by dropwise addition of 0.1M NaOH and particle absence was visually confirmed by light microscopy at 40× magnification. Data are shown in FIG. 9.

### Example 4

### Degradation Assays

Sodium cholate-based particles were prepared according to Method 1 in Example 1, using 15 mg gold(III) chloride. For comparison, particles were prepared using a double emulsion method with a reducing agent the reduce the gold ions to gold nanoparticles, which serve as a template for particle formation (see International Patent Publication No. WO 2021/011753). After preparation, the particles were suspended in deionized water at a concentration of 10 mg particles/mL water, were well mixed via vortexing, and were continuously agitated on a rotating platform at 37° C. At 24-hour intervals, samples were centrifuged at 15,000 rpm for 5 minutes and the supernatant was collected prior to resuspending particles in deionized water to the original volume. The collected supernatants were analyzed for released cholate molecules using a Bile Acid Assay Kit (Sigma-Aldrich catalog no. MAK309).

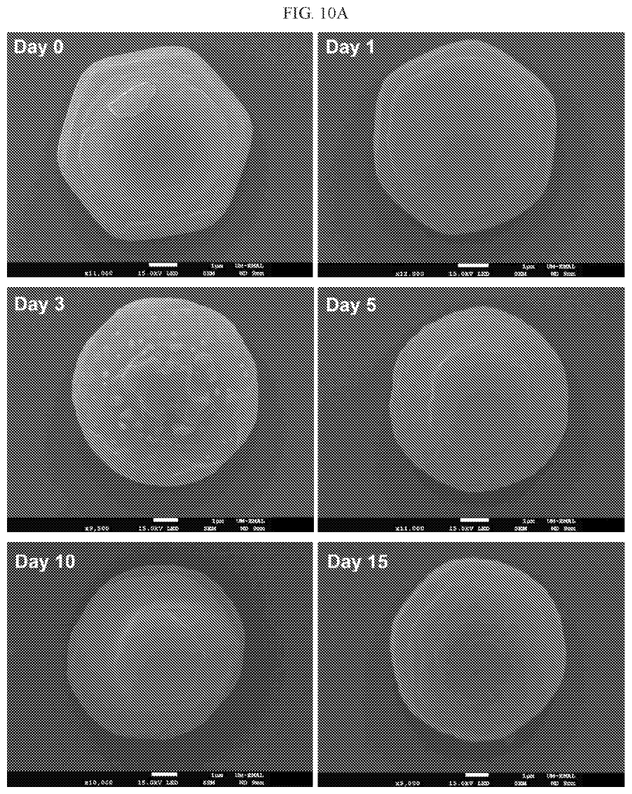

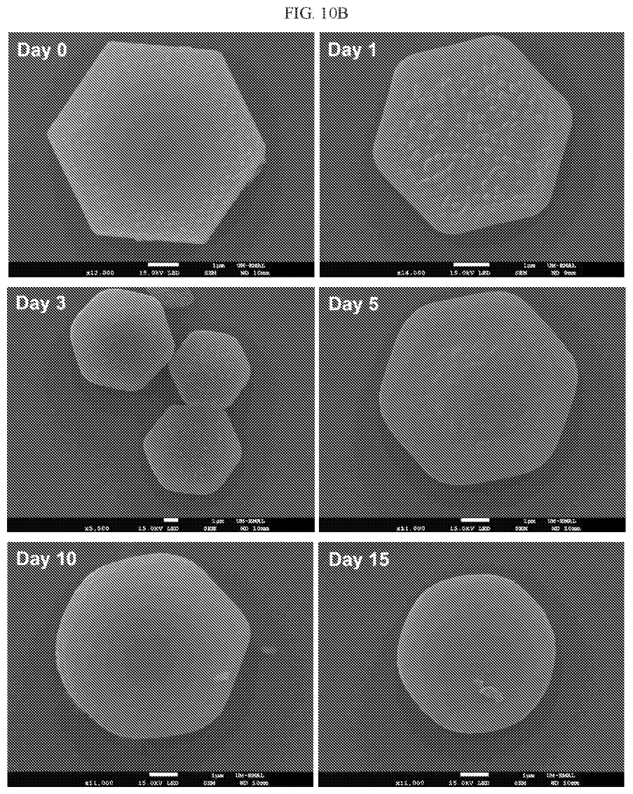

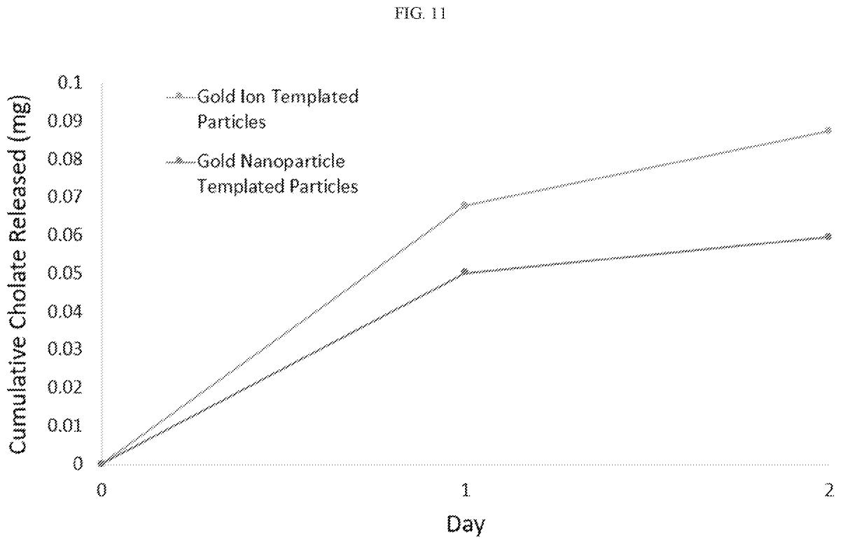

Particles were collected and imaged by SEM on days 0, 1, 3, 5, 10, and 15. Images are shown in FIGS. 10A-10B, with FIG. 10A showing images of cholate/gold. ion particles prepared according to Method 1 in Example 1, and FIG. 10B showing images of cholate/gold(0) nanoparticle particles prepared according to WO 2021/011753. The images show that the particles have similar degradation characteristics. In FIG. 10A, magnifications are 11000×, 12000×, 9500×, 11000×, 10000×, and 9000× for days 0, 1, 3, 5, 10, and 15, respectively. In FIG. 10B, magnifications are 12000×, 14000×, 5500×, 11000×, 11000×, and 11000× for days 0, 1, 3, 5, 10, and 15, respectively.

Data showing release of cholate salt from the degraded particles is shown in FIG. 11. The particles fabricated according to Method 1 disclosed herein (with gold(III) ions) were shown to release active cholate molecules in a similar manner and at a similar order of magnitude when compared to the previously-characterized gold(0) nanoparticle templated cholate particles.

### Example 5

### Detection of Gold Nanoparticles

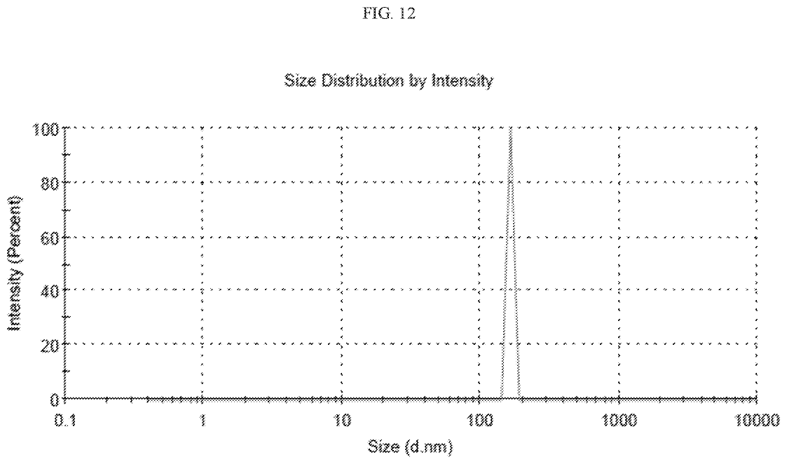

To compare cholate particles fabricated according to methods disclosed herein (Method 1 according to Example 1) to cholate particles comprising gold nanoparticles (e.g., particles disclosed in WO 2021/011753), both particles were degraded by suspending 20 mg of particles in 2 mL of 95% ethanol. The sample was well mixed to ensure all particles had degraded completely, and then centrifuged at 15,000 rpm for 10 minutes to isolate any gold nanoparticles that might have been released. The supernatant was removed, and the sample was resuspended in water and pipetted up and down to help resuspend any nanoparticles. The sample was then vortexed for 30 seconds to mix well. The cleaning steps were repeated again, and then the sample was pipetted into a cuvette for analysis by dynamic light scattering (DLS). No particles were detected in the sample of the degraded particles prepared according to Method 1 in Example 1,confirming the absence of any gold nanoparticles. By contrast, gold nanoparticles were detected in the sample prepared according to WO 2021/011753. The DLS readout for this sample is shown in FIG. 12, showing a sharp peak at approximately 170 nm. This peak corresponds to the diameter of particles present in the solution, indicating the presence of gold nanoparticles in this size range.

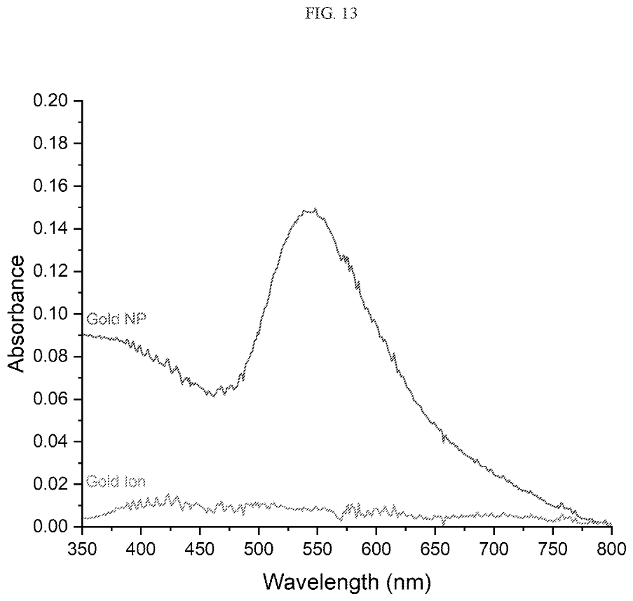

As an additional method, deoxycholate particles fabricated by each method were also degraded by dropwise addition of 0.1M NaOH and particle absence was visually confirmed by light microscopy at 40× magnification. The absorbance of the both degraded particles was measured by UV-visible spectrometry. The UV-visible spectrometry data for these samples is shown. in FIG. 13. No gold nanoparticles were detected in the sample of the degraded particles prepared according to Method 1 in Example 1, since there was no significant peak between 350 nm and 800 nm wavelengths, confirming the absence of any gold nanoparticles. By contrast, gold nanoparticles were detected with a peak at 548 nm in the sample prepared according to WO 2021/011753.

### Example 6

### Detection of Gold Ions

To confirm the presence of gold ions in the particle matrices of particles prepared according to Example 1 (e.g., by Method 1), particles were degraded by suspending them in 250 μL deionized water, and then adding 1 M MOH in 1 μL increments to fully dissolve the particles. Samples were thoroughly mixed via vortexing after each addition of NaOH, 44 μL of the NaOH solution were required to fully degrade the sample such that no particles were visible on a light microscope at 40× magnification. The degradation products were then used in a gold nanoparticle production process. If gold(III) ions are present in the degradation solution, addition of a reducing agent and heat should result in gold nanoparticle formation. 1 wt % gold ions in the particle matrix was assumed, and 17.6 mg particles were subject to degradation (thus assuming 0.176 nag gold ions and 17.424 mg cholate). For comparison, separate solutions were prepared with 17.424 mg cholate and from 0.05 to 0.5 mg gold ions in water (specifically 0.05 mg, 0.1 mg, 0.25 mg, 0.3 mg, and 0.5 mg). In each sample, gold ions were reduced to gold nanoparticles by adding 0.94 mg sodium citrate as a reducing agent, and heating at 80° C. for 10 minutes. The total volume for each sample was 1.89 mL.

Images of the samples are provided in FIG. 13. The color of solution before and after heating was used as an indicator of gold nanoparticle formation, with a color change from colorless to deep red indicating reduction of gold ions to gold nanoparticles. Before heating, no color change was observed, indicating the lack of nanoparticle formation in the absence of heat (as expected). After heating, the solutions developed a red color, with deeper colors developing for the comparison samples having higher initial gold(III) ion concentrations. The sample containing degraded particles prepared according to Method 1 in Example 1 does show development of red color, indicating nanoparticle formation, thus confirming the presence of gold(III) ions in the solution resulting from the degradation of the particles.

### Example 7

### Cell Lysis Assays

Human umbilical vein endothelial cells (HUVECs) will be cultured into 12-well plates which were pre-treated with gelatin, glutaraldehyde, and glycine as previously described (Charoenphol et al. Biomaterials. 2010; 31(6):1392-1402). After reaching confluency, the cells will be incubated with 1 ml of either bile acid solutions (e.g., cholate and deoxycholate solutions) of different concentrations, or a suspension of composite particles of known concentrations for different time-points at 37° C. and 5% CO₂. After the desired time-point, the treatment solution/suspension will be removed from the wells, the cells will be washed with warm 1× phosphate buffered saline (PBS) and incubated with 1 ml of the 1:25 dilution of MIS assay cell titer (Promega, 1) in 1×PBS at 37° C. and 5% CO₂ for 2 hours until the appearance of the orange color in the untreated control wells. The absorbance will be then measured at 490 nm, and each condition will be repeated in triplicate. The percentage of the cell viability will be quantified via subtracting the background cell titer absorbance from the absorbance of the desired point and dividing it by the average signal of the untreated cells after subtracting the background.

### Example 8

### Beef Fat Lysis Assays

The capability of the particle formulation to kill fat cells was tested by incubating them with beef fat tissue. 0.15 g beef fat tissue was incubated with 1 mg/mL deoxycholate particles in 1×PBS; the particles were prepared by different methods, including gold nanoparticle containing particles (see WO 2021/011753), gold ion-containing particles, and particles fabricating using either HCl or salicylic acid. 1×PBS was used as a negative control, and a 1% solution of sodium deoxycholate 1×PBS was used as a positive control. The mixtures were incubated at 37° C. on a rotator for 3 hours.

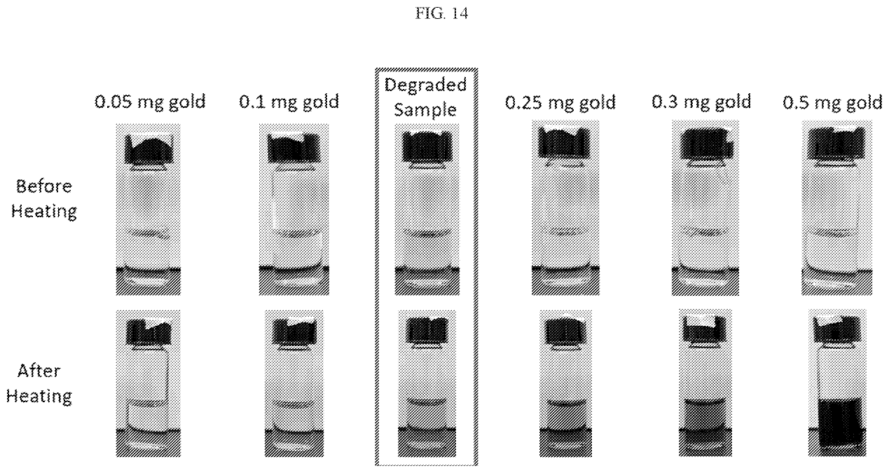

Fat lysis was qualitatively observed by the presence of lipid droplets in the sample tubes. Images of the tubes, with arrows pointing to visible droplets, are shown in FIGS. 14A and 14B. No lipid droplets were observed in the PBS control tubes.

### Example 9

### Additional Lysis Assays

The capability of the particle formulation to kill fat cells will be tested by incubating them with primary subcutaneous human adipocytes. The primary subcutaneous human adipocytes cultured in 96-well plates will be purchased from a commercial supplier (e.g., Zen-Bio, Research Triangle, NC). Upon arrival, 150 μl of the media will be removed from each well, and the cells will be incubated at 37° C. and 5% CO₂. For the lysis assays, 150 μl of the salt solution or particle suspension of the known concentration in FBS free UNE medium will be incubated for a known time-point. Afterward, the treatment solution will be aspirated, the cells will be washed with warm PBS, and 150 μl of a 1:25 dilution in 1×PBS of MIS assay cell titer (purchased from Promega Corporation, Madison, WI) will be added to each well. Plates will be incubated at 37° C. and 5% CO₂ for 3 hours or until the appearance of the orange color in the untreated control wells and the absorbance will be measured at 490 nm. The cell viability will be quantified via subtracting the background cell titer absorbance from the absorbance of the desired point and dividing it by the average signal of the untreated cells after subtracting the background.

For the control experiments, the cells will be incubated with different concentrations of bile acid (e.g., sodium cholate and sodium deoxycholate) in RPMI media.

### Example 10

### In Vivo Lysis of Mouse Adipose Tissue

In vivo lipolysis assays will be performed via subcutaneous injection of rhodamine-loaded composite particles (e.g., deoxycholate particles) into the inguinal fat pads of genetically obese mice alongside the salt solution and vehicle control. Genetically obese mice will be anesthetized using isoflurane, shaved, and subcutaneously injected with a suspension of rhodamin.e-loaded particles (e.g., deoxycholate particles) or a solution of cholate or deoxycholate salt in saline (25 mg/mL), or vehicle control into their right inguinal fat pad. 100 μL of pure saline will be injected into the left fat pad of the animals as the control. The weight and appearance of the animals will be tracked over the course of two weeks. One group of the animals will receive a second dosage of particles on Day 7. After 14 days, the animals will be euthanized, the right and left fat pad of the animals were removed, weighed, and fixed in 10% formalin solution overnight. Histology slides of the samples will be prepared via paraffin embedding and standard hematoxylin and eosin staining. The histology slides will be analyzed by blindfolded physicians and the digital scans will be analyzed using QuPath software.

### Example 11

### Cancer Cell Lysis Assays

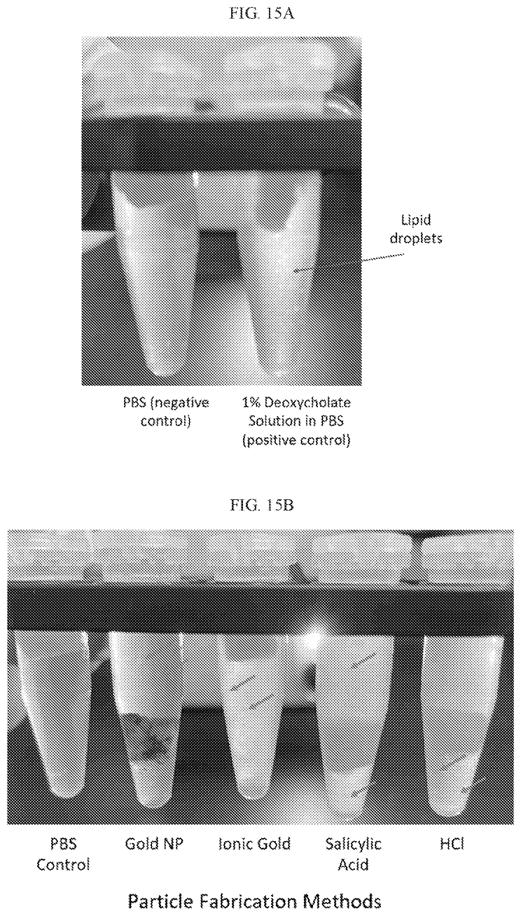

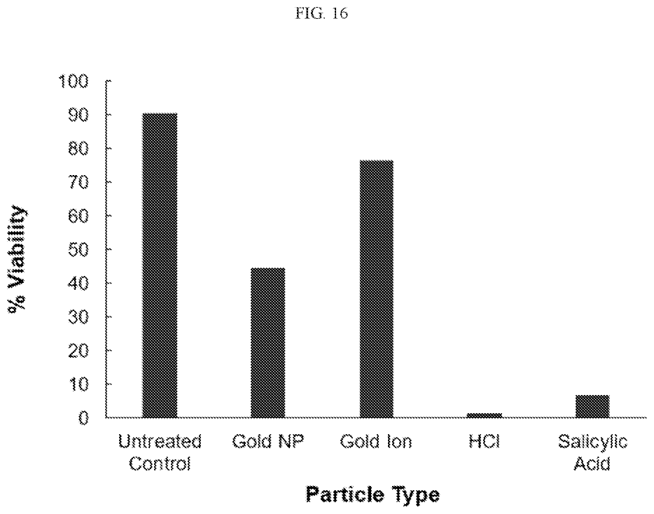

HT-29 colon cancer cells (ATCC® HTB38™) were purchased from American Type Culture Collection (ATCC) and cultured in 24-well plates until reaching confluency. Each well was incubated with a specified concentration of deoxycholate microparticles (1000 μM deoxycholate/well) in 1000 μl of McCoy's 5A media for 24 hours. 1000 μM concentrations were theoretical maximums assuming all particles dissolved immediately in well. All particle types—including gold nanoparticle containing particles (see WO 2021/011753), gold ion-containing particles, and particles fabricating using either HCl or salicylic acid—were run alongside an untreated control. Afterwards, all the wells were washed with warm PBS and their viability was quantified using Annexin Wpropidium iodide (PI) staining and flow cytometry. The percentage of the cell viability was quantified via determining which percentage of cells showed neither Annexin V staining (an indicator of cellular apoptosis) nor PI staining (necrosis or late stage apoptosis). Data are shown in FIG. 15, and show reductions in cell viability particularly for the acid-containing particles.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term. “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composite particle comprising a compound having a steroid core structure, or a salt or ester thereof, wherein the composite particle has a length in at least one dimension of at least 100 nm.

2. The composite particle of claim 1, wherein the composite particle further comprises transition metal ions.

3. The composite particle of claim 2, wherein the transition metal ions are selected from gold, silver, copper, platinum, palladium, nickel, and iron ions.

4. The composite particle of claim 3, wherein the transition metal ions are selected from gold, silver, copper, and iron ions.

5. The composite particle of claim 3, wherein the transition metal ions are Au(III) ions or Fe(II) ions.

6. The composite particle of claim 1, wherein the composite particle further comprises an acid.

7. The composite particle of claim 1, wherein the composite particle further comprises an acid.

8. The composite particle of claim 7, wherein the acid is selected fiom hydrochloric acid and salicylic acid.

9. The composite particle of any one of claims 1-8, wherein the compound having a steroid core structure is selected from the group consisting of testosterone, exemestane, formestane, mesterolone, fluoxymesterone, methyltestosterone, oxandrolone, oxymetholone, mestranol, norethindrone, danazol, gestrinone, levonorgestrel, lynestrenol, norgestrel, desogestrel, etonogestrel, tibolone, ethynodiol, cyproterone, megestrol, abiraterone, dienogest, mifepristone, drospirenone, spironolactone, estradiol, polyestradiol, estramustine, estrone, estropipate, progesterone, dydrogesterone, hydroxyprogesterone, medroxyprogesterone, segesterone, norelgestromin, norgestirnate, cortisol, cortisone, fluorometholone, difluprednate, fludrocortisone, fluocinolone, loteprednol, methylprednisolone, prednicarbate, prednisolone, prednisone, triamcinolone, alclometasone, betamethasone, clobetasol, clobetasone, clocortolone, desoximetasone, dexamethasone, diflorasone, difluoconolone, fluticasone, halometasone, rnornetasone, rimexolone, amcinonide, budesonide, ciclesonide, deflazacort, desonide, flunisolide, fluocinonide, halcinonide, cholesterol, estradiol, hydrocortisone, diflucortolone, boldenone, nandrolone, altrenogest, stanozolol, osaterone, estriol, aglepristone, trilostane, flumethasone, deoxycorticosterone, alfaxalone, desoxycorticosterone, and isoflupredone, or a salt or an ester thereof, or any combination thereof.

10. The composite particle of any one of claims 1-8, wherein the compound having a steroid core structure is a bile acid.

11. The composite particle of claim 10, wherein the bile acid is selected from cholic acid, deoxycholic acid, chenodeoxycholic acid, lithocholic acid, glycocholic acid, taurocholic acid, glycodeoxycholic acid, taurodeoxycholic acid, glycochenodeoxycholic acid, taurochenodeoxycholic acid, glycolithocholic acid, taurolithocholic acid, ursodeoxycholic acid, glycoursodeoxycholic acid, tauroursodeoxycholic acid, and obeticholic acid.

12. The composite particle of claim 10, wherein the bile acid is selected from cholic acid and deoxycholic acid.

13. The composite particle of any one of claims 1-8, wherein the compound having a steroid core structure is a salt of a bile acid.

14. The composite particle of claim 13, wherein the salt of the bile acid is selected from sodium cholate, sodium deoxycholate, sodium ursodeoxycholate, and sodium chenodeoxycholate.

15. The composite particle of any one of claims 1-8, wherein the compound having a steroid core structure is a corticosteroid compound or a salt or ester thereof.

16. The composite particle of claim 15, wherein the corticosteroid compound is selected from hydrocortisone, dexamethasone, beclomethasone, ciclesonide, clobetasol, clobetasone, desonide, desoxymethasone, desoxycorticosterone, dichlorisone, diflorasone, diflucortolone, fluclarolone, fludrocortisone, flumethasone, fluocinolone, fluocinonide, flucortine, fluocortolone, fluprednidene, flurandrenolone, halcinonide, halometasone, methylprednisolone, triamcinolone, cortisone, cortodoxone, flucetonide, fluradrenalone, medrysone, alclometasone, amciafel, amcinafide, amcinonide, betamethasone, budesonide, chlorprednisone, clocortelone, clescinolone, difluprednate, flucloronide, flunisolide, fluoromethalone, fluperolone, fluprednisolone, hydrocortamate, meprednisone, mometasone, paramethasone, prednisolone, prednisone, prednicarbate, and tixocortol, or a salt or an ester thereof.

17. The composite particle of claim 16, wherein the corticosteroid compound is selected fro dexamethasone, methylprednisolone, and hydrocortisone, or a salt or an ester thereof.

18. The composite particle of any one of claims 1-17, wherein the composite particle has a length in at least one dimension of at least 1 μm.

19. The composite particle of any one of claims 1-18, wherein the particle has a hexagonal prism shape.

20. The composite particle of claim 19, wherein the hexagonal prism has a diagonal length of 2.5 μm to 10 μm.

21. The composite particle of claim 19 or claim 20, wherein the hexagonal prism has a height of 2.5 μm to 6.5 μm.

22. The composite particle of any one of claims 1-18, wherein the particle has a rod shape.

23. The composite particle of claim 22, wherein the rod has a length of 2.5 μm to 100 μm.

24. The composite particle of claim 22 or claim 23, wherein the rod has a length of 10 μm to 50 μm.

25. The composite particle of any one of claims 1-24, wherein the particle consists essentially of: (i) the compound having a steroid core structure or salt or ester thereof, and (ii) transition metal ions and/or an acid.

26. The composite particle of any one of claims 1-25, wherein the particle is essentially free of transition metal nanoparticles.

27. A composition comprising a plurality of composite particles of any one of claims 1-26.

28. The composition of claim 27, further comprising a pharmaceutically acceptable carrier.

29. A method of making a plurality of composite particles of any one of claims 1-28, comprising:
(a) providing a first solution comprising a transition metal salt in ethyl acetate;
(b) mixing the first solution with water to form a first emulsion;
(c) mixing the first emulsion with a second solution, wherein the second solution comprises a compound having a steroid core structure or a salt or ester thereof, to form a final mixture and thereby form the composite particles.

30. A method of making a plurality of composite particles of any one of claims 1-28, comprising:
(a) providing a first solution comprising a compound having a steroid core structure or a salt or ester thereof in ethyl acetate;
(b) mixing the first solution with a transition metal salt to form a second solution;
(c) mixing the second solution with water to form a first emulsion;
(d) mixing the first emulsion with a solution of a compound having a steroid core structure or a salt or ester thereof in water to form a final mixture and thereby form the composite particles.

31. A method of making a plurality of composite particles of any one of claims 1-28, comprising:
(a) providing a first solution comprising a transition metal salt in water;
(b) mixing the first solution with ethyl acetate to form a first emulsion;
(c) mixing the first emulsion with a solution of a compound haying a steroid core structure or a salt or ester thereof in water to form a final mixture and thereby form the composite particles.

32. A method of making a plurality of composite particles of any one of claims 1-28, comprising:
(d) providing a first solution comprising an acid, a compound having a steroid core structure or a salt or ester thereof, and water;
(e) mixing the first solution with ethyl acetate to form a first emulsion;
(f) mixing the first emulsion with a solution of an acid in water to form a final mixture and thereby form the composite particles.

33. A method of making a plurality of composite particles of any one of claims 1-28, comprising:
(d) providing a first solution comprising an acid in ethyl acetate;
(e) mixing the first solution with a second solution comprising a compound haying a steroid core structure or a salt or ester thereof and water, to form a first emulsion;
(f) mixing the first emulsion with a third solution comprising a compound having a steroid core structure or a salt or ester thereof and water, to form a final mixture and thereby form the composite particles.

34. The method any one of claims 29-33, wherein the compound having a steroid core structure is a bile acid or a salt or ester thereof.

35. The method of claim 34, wherein the compound having a steroid core structure is selected from sodium cholate, sodium deoxycholate, sodium ursodeoxycholate, and sodium chenodeoxycholate.

36. The method any one of claims 29-33, wherein the compound having a steroid core structure is a corticosteroid compound or a salt or an ester thereof.

37. The method of claim 36, wherein the corticosteroid compound is selected from dexamethasone, methylprednisolone, and hydrocortisone, or a salt or an ester thereof.

38. The method of any one of claims 29-31, wherein the transition metal salt is selected from a gold(III) salt, a silver(I) salt, a copper(II) salt, a platinum(II) salt, a palladium(II) salt, a nickel(II) salt, an iron(II) salt, and an iron(III) salt.

39. The method of any one of claims 29-31, wherein the transition metal salt is selected from a gold(III) salt, a silver(I) salt, a copper(II) salt, and an iron(II) salt.

40. The method of any one of claims 29-32, wherein the transition metal salt is gold(III) chloride or iron(II) sulfate.

41. The method of any one of claims 29-40, further comprising a step of stirring the final mixture for about 15 seconds to about 15 minutes.

42. The method of any one of claims 29-41, further comprising removing the solvents from the final mixture.

43. The method of any one of claims 29-42, further comprising separating the composite particles from the final mixture.

44. The method of any one of claims 29-43, wherein the method is conducted entirely at ambient temperature.

45. The method of any one of claims 29-44, wherein the final mixture does not comprise a reducing agent.

46. A method of treating a liver disease or a peroxisotnal disorder in a subject in need of treatment, comprising administering to the subject a therapeutically effective amount of a composition of claim 27 or claim 28.

47. The method of claim 46, wherein the liver disease is a bile acid synthesis disorder or primary biliary cholangitis.

48. The method of claim 46, wherein the liver disease is a bile acid synthesis disorder due to a single enzyme defect.

49. The method of claim 46, wherein the peroxisornal disorder is a Zellweger spectrum disorder.

50. A method of non-surgical removal of a localized fat deposit in a subject, comprising contacting the deposit with an effective amount of a composition of claim 27 or claim 28.

51. A method of reducing a subcutaneous fat deposit in a subject in need thereof, comprising administering locally to the subcutaneous fat deposit in the subject an effective amount of a composition of claim 27 or claim 28.

52. A method of treating cancer in a subject in need thereof, comprising administering to the subject a therapeutically effective amount of a composition of claim 27 or claim 28.

53. The method ol claim 52, wherein the cancer is selected from colorectal cancer and gastric cancer.

54. A method of reducing the proliferation of cancer cells, comprising contacting the cells with an effective amount of a composition of claim 27 or claim 28.

55. The method of claim 54, wherein the cancer cells are selected from colorectal cancer cells and gastric cancer cells.

56. A method of treating a disorder selected from the group consisting of endocrine disorders, rheumatic disorders, collagen diseases, dermatologic diseases, allergic states, ophthalmic diseases, respiratory diseases, hematologic disorders, neoplastic diseases, gastrointestinal diseases, nervous system disorders, inflammatory disorders, and renal diseases, comprising administering to the subject a therapeutically effective amount of a composition of claim 27 or claim 28.

57. Use of a particle or composition of any cif claims 1-28.

58. Use of a particle or composition of any of claims 1-28 for removal of a localized fat deposit.

59. Use of a particle or composition of any of claims 1-28 for treating a disease selected fioni liver diseases, a peroxisomal disorder, cancer, endocrine disorders, rheumatic disorders, collagen diseases, dermatologic diseases, allergic states, ophthalmic diseases, respiratory diseases, hematologic disorders, neoplastic diseases, gastrointestinal diseases, nervous system disorders, inflammatory disorders, and renal diseases.
